(19) United States
(12) Patent Application Publication
(10) Pub. No.: US 2024/0404707 A1
(43) Pub. Date: Dec. 5, 2024

(54) MACHINE LEARNING-BASED SYSTEMS AND METHODS FOR PREDICTING LIVER CANCER RECURRENCE IN LIVER TRANSPLANT PATIENTS

(71) Applicant: UNIVERSITY OF PITTSBURGH-OF THE COMMONWEALTH SYSTEM OF HIGHER EDUCATION, Pittsburgh, PA (US)

(72) Inventors: Jianhua LUO, Wexford, PA (US); Aatur Dilip SINGHI, Sewickley, PA (US); Baoguo REN, Allison Park, PA (US); Chien-Cheng TSENG, Pittsburgh, PA (US); George MICHALOPOULOS, Pittsburgh, PA (US); Michael A. NALESNIK, Pittsburgh, PA (US); Michelle A. WOOD-TRAGESER, Pittsburgh, PA (US); Shuchang LIU, Pittsburgh, PA (US); Yanping YU, Wexford, PA (US)

(21) Appl. No.: 18/688,105

(22) PCT Filed: Sep. 2, 2022
(86) PCT No.: PCT/US2022/075886

§ 371 Date: Feb. 29, 2024

Related U.S. Application Data
(60) Provisional application No. 63/239,999, filed on Sep. 2, 2021.
(60) Provisional application No. 63/257,279, filed on Oct. 19, 2021.

(51) Int. Cl. G16H 50/30; G16B 25/10; G16B 40/20; G16H 50/20

(52) U.S. Cl. CPC G16H 50/30; G16B 25/10; G16B 40/20; G16H 50/20

(57) ABSTRACT

Described herein are prediction models based on the transcriptomic, exomic, and/or radiological analyses on tissue samples to predict the likelihood of the original cancer (such as Hepatocellular carcinoma (HCC)) recurrence into the liver transplant. An example computer implemented method for predicting the likelihood of liver cancer recurrence 5 into a liver transplant includes receiving gene expression data related to a liver tissue sample for a subject having a liver cancer, inputting the gene expression data into a trained machine learning model, and predicting, using the trained machine learning model, a risk of recurrence of the liver cancer in the subject after liver transplantation.

### CROSS-REFERENCE TO RELATED APPLICATIONS

This application claims the benefit of U.S. provisional patent application No. 63/239,999, filed on Sep. 2, 2021, and titled “MACHINE LEARNING-BASED SYSTEMS AND METHODS FOR PREDICTING LIVER CANCER RECURRENCE IN LIVER TRANSPLANT PATIENTS,” and U.S. provisional patent application No. 63/257,279, filed on Oct. 19, 2021, and titled “MACHINE LEARNING-BASED SYSTEMS AND METHODS FOR PREDICTING LIVER CANCER RECURRENCE IN LIVER TRANSPLANT PATIENTS,” the disclosures of which are expressly incorporated herein by reference in their entireties.

### STATEMENT REGARDING FEDERALLY SPONSORED RESEARCH OR DEVELOPMENT

This invention was made with government support under CA229262, TR001857 and DK120531 awarded by the National Institutes of Health (NIH). The government has certain rights in this invention.

### BACKGROUND OF THE INVENTION

Hepatocellular carcinoma (HCC) is the most frequent type of liver cancer, and has a general 5-year survival rate of 18% (1), with only glioblastoma and pancreatic cancer having higher mortality (2). Currently, surgical therapy of HCC remains the most effective approach to treat HCC. When HCC is localized and the liver function is adequate, tumor resection or cryoablation therapy may be a treatment option. However, liver transplant is the mainstay of HCC treatment because it treats both the HCC tumor and the underlying liver disease such as cirrhosis or alcoholic fatty liver disease. Thus, it eliminates the risk of new tumor formation from the underlying liver disease.

The first successful liver transplant was conducted in 1963 (3). Since then, the number of liver transplants applied to treat HCC has been steadily increased. Milan criteria were developed in 1996 to guide the selection of HCC patients for the liver transplant treatment by restricting the HCC single lesion <5 cm in diameter or up to 3 tumor nodules but with no tumor nodule >3 cm in diameter (4). However, Milan criteria was later viewed as too restrictive, and thus denied a large number of HCC patients the transplant treatment. Several subsequent criteria were developed to relax the criteria to include more patients into the liver transplant program (5). The latest Extended Toronto criteria include patients with any size or number of tumors if the patient is negative in systemic cancer-related symptoms or extrahepatic disease or poorly differentiated cancer based on biopsy (6). The post-transplant survival rates from these criteria ranged from 65-85% (7). One of the major considerations of selection of transplant candidates is post-transplant HCC recurrence. Based on various studies, the HCC recurrence rate was up to 20% among liver transplant patients and had a median recurrence time of 14 months after the transplant. The median post-recurrence survival time is only 12 months (8, 9). Thus, a better prediction method of HCC recurrence for the HCC liver transplant candidates is necessary to improve the clinical outcomes of HCC patients.

### SUMMARY OF THE INVENTION

Described herein are prediction models based on the transcriptomic, exomic, and/or radiological analyses on tissue samples to predict the likelihood of the original cancer recurrence into the liver transplant. For example, in some implementations described herein, prediction models based on the transcriptomic, exomic, and/or radiological analyses on HCC samples to predict the likelihood of the original HCC recurrence into the liver transplant are described.

An example computer-implemented method for predicting the likelihood of liver cancer recurrence into a liver transplant is described. The method includes receiving gene expression data related to a liver tissue sample for a subject having a liver cancer, inputting the gene expression data into a trained machine learning model, and predicting, using the trained machine learning model, a risk of recurrence of the liver cancer in the subject after liver transplantation.

Additionally, the trained machine learning model is a supervised machine learning model. For example, the trained machine learning model can be a support vector machine (SVM), a random forest model, a logistic regression model, or a k-top scoring pairs (k-TSP) model.

In some implementations, the trained machine learning model is configured to predict the risk of recurrence as a probability score. In other implementations, the trained machine learning model is configured to predict the risk of recurrence by classifying the subject into one of a plurality of categories.

Alternatively or additionally, the gene expression data includes respective gene expression levels for a top-n differentially expressed genes, where n is an integer greater or equal to 10. Optionally, n is greater than or equal to 50. In these implementations, the trained machine learning model is a random forest model. Additionally, the top-n differentially expressed genes can include one or more of HOOK1, EFCAB7, CDC7, NUF2, UBE2T, HELLS, RRM1, SYT12, KIF21A, RACGAP1, PRIM1, PTGES3, YEATS4, CCT2, PARPBP, PPP1CC, KNTC1, TMED2, CDKN3, DLGAP5, BUB1B, NUSAP1, CCNB2, KIF23, FANCI, PRC1, CDC6, TOP2A, KPNA2, NDC80, RBBP8, NARS, BUB1, TOPBP1, SMC4, NCAPG, CENPE, PLK4, CENPU, CENPQ, TTK, FBXO5, ANLN, MELK, DYNLT3, ZNF674, KIF4A, AMMECR1, ZNF449, and BRCC3.

Alternatively or additionally, the gene expression data includes respective gene expression levels for a top-q pairs of differentially expressed genes, where q is an integer greater or equal to 10. Optionally, q is 43. In these implementations, the trained machine learning model is a k-TSP model. Additionally, the top-q pairs of differentially expressed genes includes one or more of BUB1B and SSH3, MCM8 and OGDHL, NUSAP1 and FNDC4, KIF21A and RAB43, CDC7 and CNGA1, MORF4L2 and ETFB, HELLS and HAMP, PPIL1 and ZCCHC24, MELK and GALNT15, BRCC3 and CCDC69, CCT6A and ASL, CDKN3 and SYT12, RBBP8 and TMPRSS2, KIF23 and LMF1, KPNA2 and SUN2, SMC4 and FXYD1, PPP1CC and FTCD, NUCB2 and NDRG2, PARP2 and IL11RA, VBP1 and AGTR1, TOP2A and TSPAN9, KTN1 and COL18A1, NCAPG and ADAMTS13, STT3B and CD14, SEC11C and C8G, CCNA2 and ADRA1A, CENPQ and UROC1, TTK and PLCH2, FANCI and SHBG, DEK and EGR1, RFC5 and APOF, PTGES3 and TAT, SNX7 and PGLYRP2, CCT2 and PIGR, PRC1 and MGMT, NARS and MASP1, RRM1 and MGLL, TOPBP1 and CTSF, F2 and ITIH4, ANLN and ZNF674, PRIM1 and SULTIA1, RARRES2 and HRG, and CENPU and NNMT.

In some implementations, the method optionally further includes receiving mutation data related to the liver tissue sample, and inputting the mutation data into the trained machine learning model. The trained machine learning model can be a random forest model, a k-top scoring pairs (k-TSP) model, a support vector machine (SVM), or a logistic regression model. Additionally, the mutation data includes a number of somatic mutations present in the liver tissue sample from the subject. For example, in some implementations, the mutation data optionally includes a number of somatic mutations present in a top-m mutation pathways, where m is an integer greater or equal to 5. Optionally, m is 5. In these implementations, the trained machine learning model is a random forest model. Additionally, the top-m mutation pathways include GO_ENDONUCLEASE_ACTIVITY_ACTIVE_WITH_EITHER_RIBO_OR_DEOXYRIBON UCLEIC_ACIDS_AND_PRODUCING_3_PHOSPHOMONOESTERS, GO_GLUCOSE_BINDING, GO_PALMITOYL_COA_HYDROLASE_ACTIVITY, GO_PEPTIDE_N_ACETYLTRANSFERASE_ACTIVITY, and GO_DOPAMINE_BINDING. Alternatively, in other implementations, the mutation data includes a number of somatic mutations present in a top-r mutation pathway pairs, where r is an integer greater or equal to 3. Optionally, r is 3. In these implementations, the trained machine learning model is a k-top scoring pairs (k-TSP) model. Additionally, the top-r mutation pathways pairs includes GO_SYNTAXIN_BINDING and GO_N_ACYLTRANSFERASE_ACTIVITY, REACTOME_GOLGI_ASSOCIATED_VESICLE_BIOGENESIS and GO_N_ACETYLTRANSFERASE_ACTIVITY, and GO_REGULATION_OF_HORMONE_METABOLIC_PROCESS and GO_PEPTIDE_N_ACETYLTRANSFERASE_ACTIVITY.

In some implementations, the method optionally further includes receiving a radiology-based parameter related to the liver cancer, and inputting the radiology-based parameter into the trained machine learning model. Additionally, the radiology-based parameter is based on a size or number of tumor nodules associated with the liver cancer. For example, the radiology-based parameter is optionally Milan criteria. In some implementations, the trained machine learning model is a k-top scoring pairs (k-TSP) model. In other implementations, the trained machine learning model is a support vector machine (SVM), a random forest model, or a logistic regression model.

In some implementations, the method optionally further includes receiving mutation data related to the liver tissue sample and a radiology-based parameter related to the liver cancer, and inputting the mutation data and the radiology-based parameter into the trained machine learning model. Optionally, the trained machine learning model is a random forest model. Alternatively, the trained machine learning model is optionally a support vector machine (SVM), a logistic regression model, or a k-top scoring pairs (k-TSP) model.

In some implementations, the method optionally further includes providing a treatment recommendation based on the prediction. For example, the treatment recommendation is to perform a liver transplant procedure on the subject.

Alternatively or additionally, the liver cancer is hepatocellular carcinoma (HCC).

An example method for treating liver cancer is described. The method includes predicting a risk of recurrence of a liver cancer in a subject after liver transplantation as described herein, recommending the subject as a candidate for a liver transplant procedure based on the prediction, and performing the liver transplant procedure on the subject.

An example system for predicting the likelihood of liver cancer recurrence into a liver transplant is described. The system includes a trained machine learning model and a computing device including a processor and a memory, the memory having computer-executable instructions stored thereon. The computing device is configured to receive gene expression data related to a liver tissue sample for a subject having a liver cancer, input the gene expression data into the trained machine learning model, and receive a risk of recurrence of the liver cancer in the subject after liver transplantation, where the risk of recurrence is predicted by the trained machine learning model.

In some implementations, the computing device is further configured to receive mutation data related to the liver tissue sample, and input the mutation data into the trained machine learning model.

In some implementations, the computing device is further configured to receive a radiology-based parameter related to the liver cancer, and input the radiology-based parameter into the trained machine learning model.

Alternatively or additionally, in some implementations, the trained machine learning model is configured to predict the risk of recurrence as a probability score. In other implementations, the trained machine learning model is configured to predict the risk of recurrence by classifying the subject into one of a plurality of categories.

Alternatively or additionally, the trained machine learning model is a support vector machine (SVM), a random forest model, a logistic regression model, or a k-top scoring pairs (k-TSP) model.

In some implementations, the computing device is further configured to provide a treatment recommendation based on the prediction. For example, the treatment recommendation is to perform a liver transplant procedure on the subject.

Alternatively or additionally, the liver cancer is hepatocellular carcinoma (HCC).

It should be understood that the above-described subject matter may also be implemented as a computer-controlled apparatus, a computer process, a computing system, or an article of manufacture, such as a computer-readable storage medium.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1 is a diagram illustrating a machine learning model for predicting a risk of recurrence of a liver cancer in a subject after liver transplantation according to implementations described herein.

FIG. 2 is an example computing device.

FIG. 3 is a flow chart of procedures for training and validation of genome prediction model. The procedure starts with the identification of cancer cases by the year of liver transplant surgery using 2012 as the demarcation. All cases before the first half of 2012 were used the training set, while cases after the second half of 2012 were used as the testing set. The cancer areas and benign tissues from the non-liver organ of the paraffin block were needle-cored and used as “cancer” and “normal” tissues, respectively. All clinical information was blind to the researchers before the prediction.

FIGS. 4A-4F is a receiver operating characteristic (ROC) analysis of genome prediction model. FIG. 4A is training set ROC based on top 500 differentially expressed genes between recurrence and non-recurrence samples from the transcriptome sequencing using Leave-One-Out Cross-Validation (LOOCV) strategy with Random Forest method. FIG. 4B is testing set ROC based on the algorithm determined in the training set of FIG. 4A. FIG. 4C is training set ROC based on transcriptome and exome sequencing results using Random Forest method. FIG. 4D is testing set ROC based on the algorithm determined in the training set of FIG. 4C. FIG. 4E is ROC of pooled training and testing cohorts based on transcriptome sequencing using LOOCV strategy with Random Forest method. FIG. 4F is ROC of pooled training and testing cohorts based on transcriptome and exome sequencings using LOOCV strategy with Random Forest method.

FIGS. 5A-5F is a Kaplan-Meier analysis of genome prediction model. FIG. 5A is a training set Kaplan-Meier analysis based on 500 differentially expressed genes from the transcriptome sequencing using LOOCV strategy with Random Forest method. FIG. 5B is a testing set Kaplan-Meier analysis based on the algorithm determined in the training set of FIG. 5A. FIG. 5C is a training set Kaplan-Meier analysis based on transcriptome and exome sequencing results using Random Forest method. FIG. 5D is a testing set Kaplan-Meier analysis based on the algorithm determined in the training set of FIG. 5C. FIG. 5E is a Kaplan-Meier analysis of pooled training and testing cohorts based on transcriptome sequencing using LOOCV strategy with Random Forest method. FIG. 5F is a Kaplan-Meier analysis of pooled training and testing cohorts based on transcriptome and exome sequencings using LOOCV strategy with Random Forest method.

FIGS. 6A-6F is a ROC analysis of Milan criteria with the genome prediction model. FIG. 6A is a ROC analysis based on Milan criteria in the training set. FIG. 6B is a ROC analysis based on Milan criteria in the testing set. FIG. 6C is a ROC analysis based on Milan criteria in the combined training and testing sets. FIG. 6D is a ROC analysis of the training set based on Milan/transcriptome k-top scoring pairs (k-TSP) prediction model using LOOCV. FIG. 6E is a ROC analysis of the testing set based on Milan/transcriptome k-TSP prediction algorithm determined in FIG. 6D. FIG. 6F is a ROC analysis of the combined training and testing sets based on Milan/transcriptome k-TSP prediction model using LOOCV.

FIGS. 7A-7F is a Kaplan-Meier analysis of Milan criteria with the genome prediction model. FIG. 7A is a Kaplan-Meier analysis based on Milan criteria in the training set, FIG. 7B is a Kaplan-Meier analysis based on Milan criteria in the testing set, and FIG. 7C is a Kaplan-Meier analysis based on Milan criteria in the combined training and testing sets. FIG. 7D is a Kaplan-Meier analysis of the training set based on Milan/transcriptome k-TSP prediction model using LOOCV. FIG. 7E is a Kaplan-Meier analysis of the testing set based on Milan/transcriptome k-TSP prediction algorithm determined in FIG. 7D. FIG. 7F is a Kaplan-Meier analysis of the combined training and testing sets based on Milan/transcriptome k-TSP prediction model using LOOCV.

FIGS. 8A-8C illustrate transcriptomic alteration related to recurrence and mutation pathways of HCC samples. FIG. 8A depicts Hierarchical clustering of HCC samples based on top 500 differential expression genes between non-recurrence and recurrence HCC samples. FIG. 8B is a Heat map of five signaling pathways based on the differential mutation numbers in the pathways between non-recurrence and recurrence samples. FIG. 8C depicts Gene expression alterations and connections based on Gene ontology analysis.

FIGS. 9A-9F is a ROC analysis of Milan criteria with the transcriptome/mutation pathways RF prediction model. FIG. 9A is a ROC analysis based on Milan criteria in the training set, FIG. 9B is a ROC analysis based on Milan criteria in the testing set, and FIG. 9C is a ROC analysis based on Milan criteria in the combined training and testing sets. FIG. 9D is a ROC analysis based on Milan/transcriptome/mutation pathways RF prediction model in the training set. FIG. 9E is a ROC analysis of testing set based on the algorithm developed from FIG. 9D. FIG. 9F is a ROC analysis based on Milan criteria and the transcriptome/mutation pathways RF prediction model in the combined training and testing sets.

FIGS. 10A-10F is a Kaplan-Meier analysis of Milan criteria with the transcriptome/mutation pathways RF prediction model. FIG. 10A is a Kaplan-Meier analysis based on Milan criteria in the training set, FIG. 10B is a Kaplan-Meier analysis based on Milan criteria in the testing set, and FIG. 10C is a Kaplan-Meier analysis based on Milan criteria in the combined training and testing sets. FIG. 10D is a Kaplan-Meier analysis based on Milan/transcriptome/mutation pathways RF model in the training set. FIG. 10E is a Kaplan-Meier analysis of the testing set based on the algorithm developed from FIG. 10D. FIG. 10F is a Kaplan-Meier analysis based on Milan/transcriptome/mutation pathways RF prediction model in the combined training and testing sets.

FIGS. 11A-11F is a ROC analysis of k-TSP genome prediction model. FIG. 11A is a Training set ROC analysis based on top gene pairs from the transcriptome sequencing using LOOCV strategy with k-TSP method. FIG. 11B is a Testing set ROC analysis based on the algorithm determined in the training set of FIG. 11A. FIG. 11C is a Training set ROC based on transcriptome and exome sequencing results using k-TSP method. FIG. 11D is a Testing set ROC based on the algorithm determined in the training set of FIG. 11C. FIG. 11E is a ROC based on transcriptome sequencing, exome sequencing and Milan criteria on the training set using LOOCV strategy with k-TSP method. FIG. 11F is a Testing set ROC based on the algorithm determined in the training set of FIG. 11E.

FIGS. 12A-12F is a Kaplan-Meier analysis of genome prediction model. FIG. 12A is the training set Kaplan-Meier analysis based on top gene pairs from the transcriptome sequencing using LOOCV strategy with k-TSP method. FIG. 12B is the testing set Kaplan-Meier analysis based on the algorithm determined in the training set of FIG. 12A. FIG. 12C is the training set Kaplan-Meier analysis based on transcriptome and exome sequencing results using k-TSP method. FIG. 12D is the testing set Kaplan-Meier analysis based on the algorithm determined in the training set of FIG. 12C. FIG. 12E is a Kaplan-Meier analysis based on transcriptome sequencing, exome sequencing and Milan criteria on the training set using LOOCV strategy with k-TSP method. FIG. 12F is the testing set Kaplan-Meier analysis based on the algorithm determined in the training set of FIG. 12E.

FIG. 13 illustrates samples plots based on the scores generated by Milan/transcriptome/mutation pathways k-TSP model. LOOCV was performed. Incorrectly predicted samples were marked by cross-boxes, while correctly predicted samples were marked by solid circles. Red indicates recurrence samples. Blue indicates non-recurrence samples. TN—true negative; FN—false negative; TP—true positive; FP—false positive.

FIGS. 14A-14B is a principle component analysis (PCA) of 64 HCC samples based on top 500 ranked differentially expressed genes between recurrence and non-recurrence samples. FIG. 14A is a PCA of 38 samples from the training set. FIG. 14B is a PCA of 26 samples from the testing set. “Recur” shading indicates recurrence samples. “Non-recur” shading indicates non-recurrence samples.

FIGS. 15A-15C illustrates transcriptomic alteration related to recurrence and mutation pathways of HCC samples based on k-TSP model. FIG. 15A is a Hierarchical clustering of HCC samples based on 47 pairs of genes. FIG. 15B is a Heat map of three pairs of signaling pathways based on the mutation numbers in the samples. FIG. 15C depicts Gene expression alteration and connections based on Gene ontology analysis.

FIGS. 16A-16B are graphs illustrating pathway ranking by based on the adjusted p-value of the pathway impacted by expression alterations. FIG. 16A depicts Pathway ranking based on transcriptome RF analysis. FIG. 16B depicts Pathway ranking based on transcriptome k-TSP analysis.

FIG. 17 is Table 1, which includes clinical features of HCC transplant patients in an example described herein.

FIG. 18 is Table 2, which includes multiple cancer nodule predictions from HCC patients in an example described herein.

FIG. 19 is Supplemental Table 1, which includes machine learning model training and testing validation results from an example described herein.

FIG. 20 is Supplemental Table 2, which includes mutation counts for training sample sets from an example described herein.

FIG. 21 is Supplemental Table 3, which includes differential gene expression scores of 500 genes for a random forest prediction model from an example described herein.

FIG. 22 is Supplemental Table 4, which includes differential gene expression scores of 86genes for a k-TSP prediction model from an example described herein.

FIG. 23 is Table 3, which illustrates the top-500 differentially expressed genes for random forest modelling according to an example implementation.

FIG. 24 is Table 4, which illustrates the top-43 pairs of differentially expressed genes for k-TSP modelling according to an example implementation.

FIG. 25 is Table 5, which illustrates the top-5 mutation pathways impacted by expression alterations for random forest modelling according to an example implementation. FIG. 26 is Table 6, which illustrates the top-3 mutation pathway pairs impacted by expression alterations for k-TSP modelling according to an example implementation. FIG. 27 is Table 7, which illustrates the genes associated with the top-5 mutation pathways shown in FIG. 25.

FIG. 28 is Table 8, which illustrates the genes associated with the top-3 mutation pathway pairs shown in FIG. 26.

### DETAILED DESCRIPTION OF THE INVENTION

Described herein are machine learning-based systems and methods for predicting a risk of recurrence of a liver cancer in a subject after liver transplantation. Optionally, the liver cancer may be hepatocellular carcinoma (HCC). As described herein, HCC is a common type of liver cancer. Additionally, HCC oftentimes occurs in subjects with underlying liver disease such as cirrhosis or alcoholic fatty liver disease. And as a consequence, liver transplantation is the mainstay treatment since it eliminates risk of new tumor formation from the underlying liver disease. Unfortunately, post-transplant HCC recurrence is still a possibility. In other words, HCC recurs in the transplanted liver in some patients who undergo liver transplantation.

Predicting recurrence of HCC in a subject post-transplant, however, is a difficult task. For example, conventional techniques for selecting HCC patients for liver transplantation such as the Milan criteria or Extended Toronto criteria have been shown to have issues including being overly restrictive and/or fail to achieve sufficient prediction accuracy.

The machine learning-based systems and methods described herein address problems associated with the conventional techniques. As described herein, the machine learning-based systems and methods are capable of accurately predicting recurrence of HCC in liver transplant patients. Such information can be used clinically to guide and deliver treatment (e.g., select candidates for liver transplantation). In some implementations, the machine learning-based systems and methods are based on a transcriptome analysis. In other implementations, the machine learning-based systems and methods are based on both transcriptome and exome analyses. In yet other implementations, the machine learning-based systems and methods are based on both transcriptome and radiology analyses. In yet other implementations, the machine learning-based systems and methods are based on transcriptome, exome, and radiology analyses. As described herein, the machine learning-based systems and methods reveal surprising findings about the altered gene expression and/or mutations that play important roles in predicting the behavior of HCC in liver transplant patients.

Although HCC is provided as an example liver cancer, this disclosure contemplates that the liver cancer may be another type including, but not limited to, cholangiocarcinoma or angiosarcoma. This disclosure contemplates predicting a post-transplantation risk of recurrence of other types of liver cancer in a subject using the machine learning-based systems and methods described herein.

### Terminology

As used in the specification and claims, the singular form “a,” “an,” and “the” include plural references unless the context clearly dictates otherwise. For example, the term “a cell” includes a plurality of cells, including mixtures thereof.

The term “about” as used herein when referring to a measurable value such as an amount, a percentage, and the like, is meant to encompass variations of ±20%, ±10%, ±5%, or ±1% from the measurable value.

“Administration” of “administering” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, or via a transdermal patch, and the like. Administration includes self-administration and the administration by another.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

As used herein, the term “comprising” is intended to mean that the systems, compositions and methods include the recited elements, but not excluding others. “Consisting essentially of” when used to define systems, compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps. Embodiments defined by each of these transition terms are within the scope of this invention.

A “control” is an alternative subject or sample used in an experiment for comparison purposes. A control can be “positive” or “negative.”

“Inhibit”, “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

The term “increased” or “increase” as used herein generally means an increase by a statically significant amount; for the avoidance of any doubt, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “reduced”, “reduce”, “suppress”, or “decrease” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The term “subject” is defined herein to include animals such as mammals, including, but not limited to, primates (e.g., humans), cows, sheep, goats, horses, dogs, cats, rabbits, rats, mice and the like. In some embodiments, the subject is a human.

The terms “treat,” “treating,” “treatment,” and grammatical variations thereof as used herein, include partially or completely delaying, alleviating, mitigating or reducing the intensity of one or more attendant symptoms of a disorder or condition and/or alleviating, mitigating or impeding one or more causes of a disorder or condition. Treatments according to the invention may be applied preventively, prophylactically, pallatively or remedially. Prophylactic treatments are administered to a subject prior to onset (e.g., before obvious signs of cancer), during early onset (e.g., upon initial signs and symptoms of cancer), or after an established development of cancer. Prophylactic administration can occur for several days to years prior to the manifestation of symptoms of a disease (e.g., a cancer).

### Example Systems

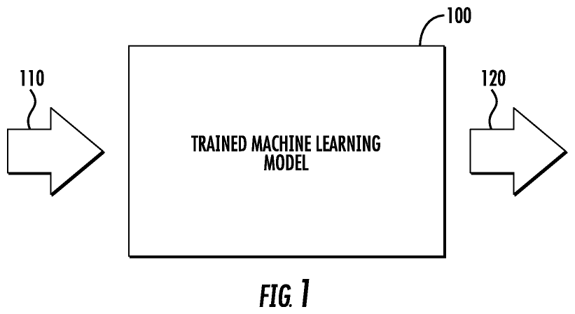

Referring now to FIG. 1, a block diagram illustrating a machine learning model 100 for predicting a risk of recurrence of a liver cancer in a subject after liver transplantation is shown. As described herein, the liver cancer is optionally HCC. In FIG. 1, the machine learning model 100 is operating in inference mode. The machine learning model 100 has therefore been trained with a data set (or “dataset”). The machine learning model 100 is sometimes referred to herein as a “trained machine learning model.” The machine learning model 100 in FIG. 1 is a supervised learning model. Supervised learning models are known in the art. A supervised learning model “learns” a function that maps an input 110 (also known as feature or features) to an output 120 (also known as target or targets) during training with a labeled data set. In some implementations, the machine learning model 100 is a regression model, i.e., it is configured to predict the subject's risk of liver cancer recurrence as a probability score. In other implementations, the machine learning model 100 is a classifier, i.e., it is configured to classify the subject into one of a plurality of categories based on the subject's risk of liver cancer recurrence. Additionally, it should be understood that the machine learning model 100 shown in FIG. 1 can include one or more machine learning models. For example, in some implementations, a plurality of features (e.g., transcriptome data, exome data, radiology data, or combinations thereof) are input into a single machine learning model. Alternatively, in other implementations, a plurality of features are input into a plurality of machine learning models (e.g., transcriptome data into a first machine learning model, exome data into a second machine learning model, etc.), and the respective outputs are then integrated (e.g., weighted and summed) to obtain the output 120 of the machine learning model 100, which includes the plurality of machine learning models.

This disclosure contemplates that the machine learning model 100 can be implemented using one or more computing devices (e.g., a processing unit and memory as described herein). In some implementations, the machine learning model is a support vector machine (SVM). In some implementations, the machine learning model is a random forest model. In some implementations, the machine learning model is a logistic regression model. In some implementations, the machine learning model is a k-top scoring pairs (k-TSP) model. It should be understood that SVM, random forest, logistic regression, and k-TSP models are provided only as example machine learning models. This disclosure contemplates that the machine learning model 100 may be another type of model including, but not limited to, linear discriminant analysis (LDA).

The machine learning model 100 is trained with a data set to maximize or minimize an objective function. In some implementations, the objective function is a cost function, which is a measure of the machine learning model's performance (e.g., error such as L1 or L2 loss). The training algorithm tunes the model's weights and/or bias to minimize the cost function. This disclosure contemplates that any algorithm that finds the maximum or minimum of the objective function can be used for training the machine learning model 100. Machine learning models and training are known in the art and are therefore not described in further detail herein.

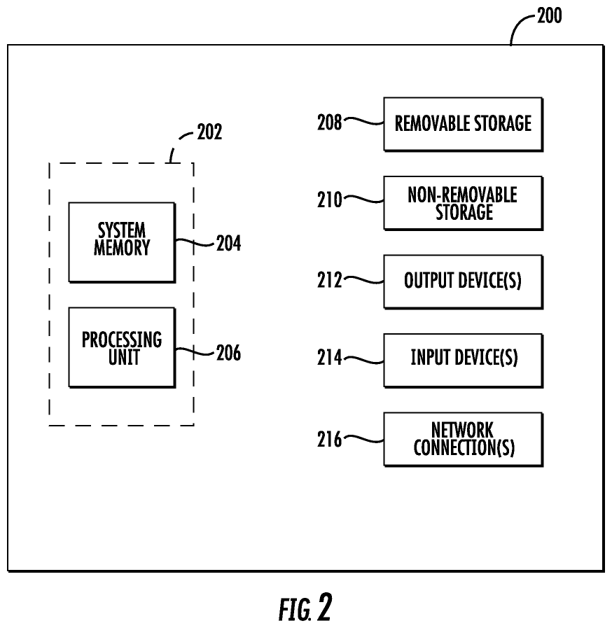

Additionally, the machine learning model 100 can optionally be operably coupled with a computing device such as computing device 200 shown in FIG. 2. For example, the machine learning model 100 and computing device can be coupled through one or more communication links. This disclosure contemplates the communication links are any suitable communication link. For example, a communication link may be implemented by any medium that facilitates data exchange between the machine learning model 100 and computing device including, but not limited to, wired, wireless and optical links. Example communication links include, but are not limited to, a local area network (LAN), a wireless local area network (WLAN), a wide area network (WAN), a metropolitan area network (MAN), Ethernet, the Internet, or any other wired or wireless link such as WiFi, WiMax, 3G, 4G, or 5G. The computing device can transmit data (e.g., the feature(s) or input 110) to the machine learning model 100 and receive data (e.g., target(s) or output 120) from the machine learning model 100.

As described above, the machine learning model 100 is trained to map the input 110 (or feature(s)) to the output 120 (or target(s)). As described below, the input 110 is gene expression data (transcriptome analysis), mutation data (exome analysis), radiology-based parameters, or combinations thereof, and the output 120 is the subject's risk of recurrence of a liver cancer after liver transplantation. For example, in some implementations described herein, the input 110 is gene expression data (transcriptome analysis), and the output 120 is the subject's risk of recurrence of a liver cancer after liver transplantation. In other implementations described herein, the input 110 is gene expression data (transcriptome analysis) and mutation data (exome analysis), and the output 120 is the subject's risk of recurrence of a liver cancer after liver transplantation. In other implementations described, the input 110 is gene expression data (transcriptome analysis) and radiology-based parameters, and the output 120 is the subject's risk of recurrence of a liver cancer after liver transplantation. In yet other implementations described herein, the input 110 is gene expression data (transcriptome analysis), mutation data (exome analysis), and radiology-based parameters, and the output 120 is the subject's risk of recurrence of a liver cancer after liver transplantation. The input 110 includes the one or more “features” that are input into the machine learning model 100, which predicts the subject's risk of recurrence of a liver cancer after liver transplantation. The subject's predicted risk is therefore the “target” of the machine learning model 100. The output 120 can be a probability score or a classification.

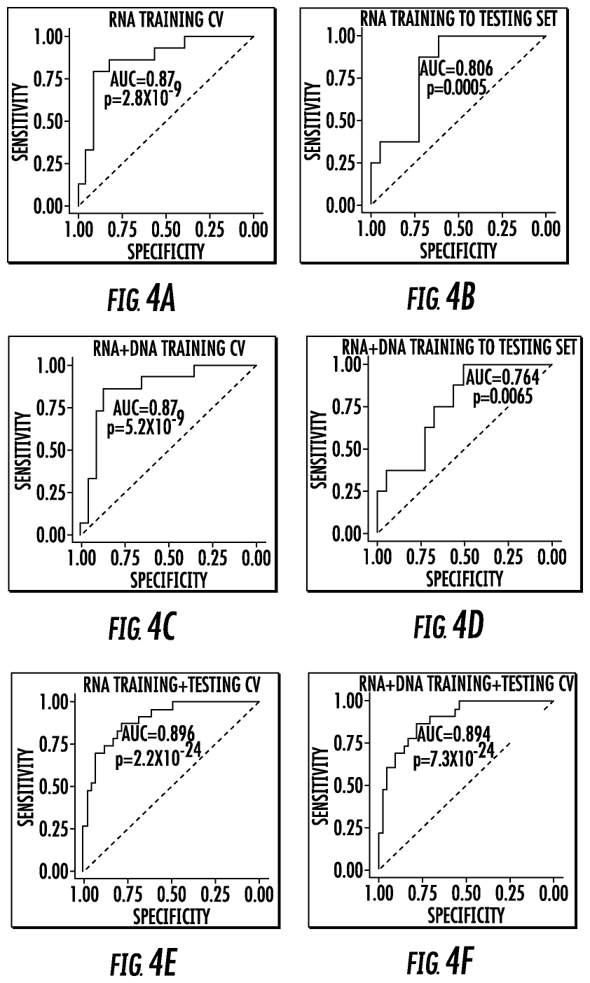

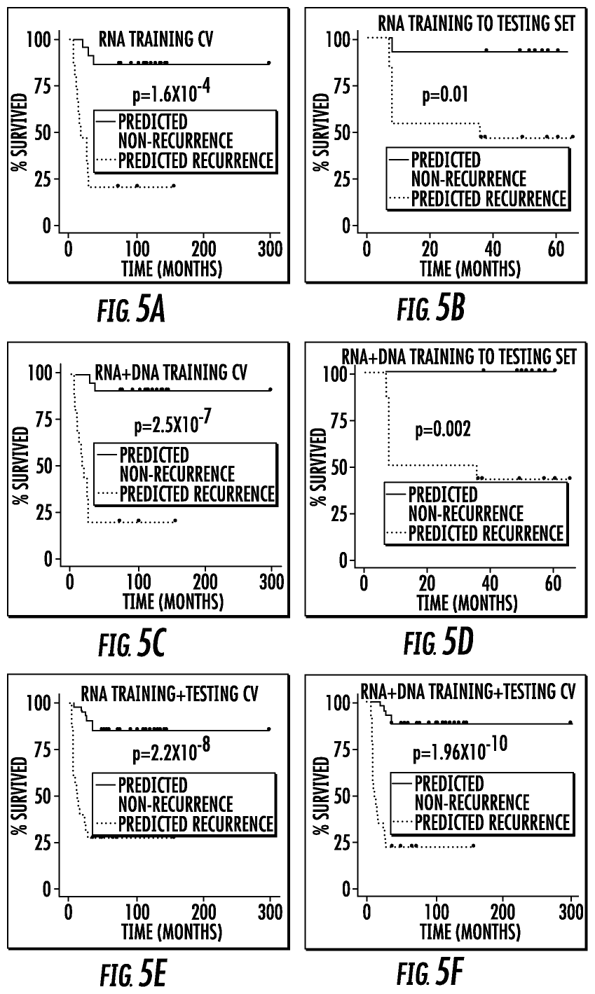

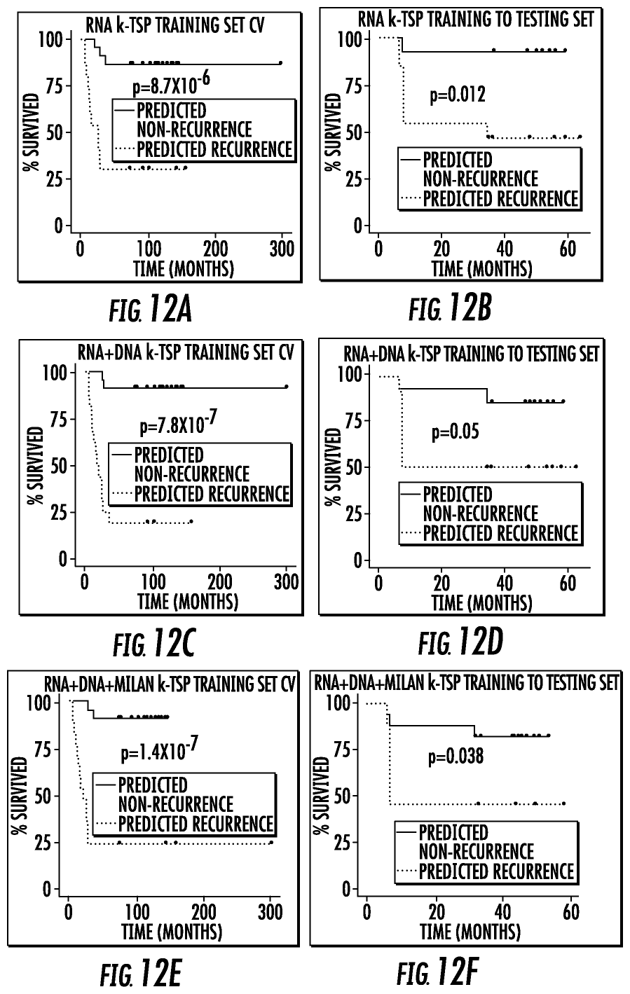

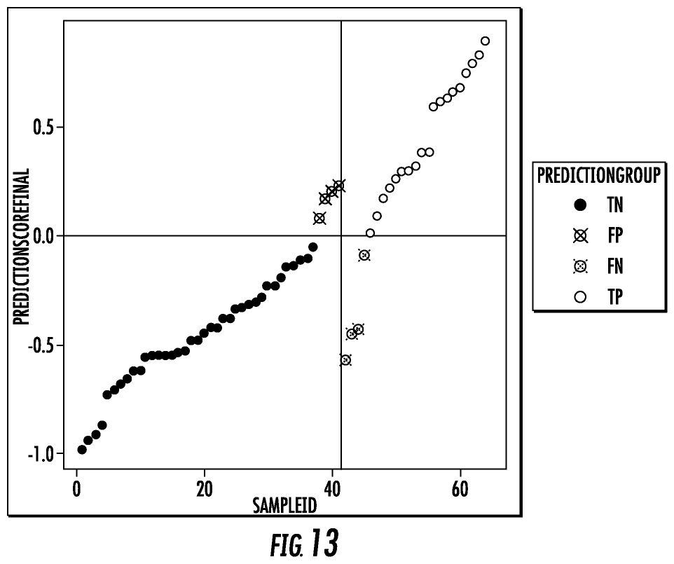

In some implementations described herein, the input 110 includes data from a transcriptome analysis. For example, the input 110 to the machine learning model 100 can include gene expression data related to a liver tissue sample. This disclosure contemplates that gene expression levels can be obtained using techniques known in the art. For example, gene expression levels can be obtained by sampling liver tissue, extracting messenger Ribonucleic acid (mRNA) from the sample, sequencing the mRNA, and quantifying the gene expression levels as compared to a control. This disclosure contemplates that an SVM, a random forest model, a logistic regression model, or a k-TSP model can be used to predict the subject's risk of recurrence of a liver cancer after liver transplantation based on features from the transcriptome analysis. For example, FIGS. 4A, 4B, 4E, 5A, 5B, and 5E illustrate analysis of a random forest prediction model according to examples described herein, and FIGS. 11A, 11B, 12A, and 12B illustrate analysis of a k-TSP prediction model according to examples described herein.

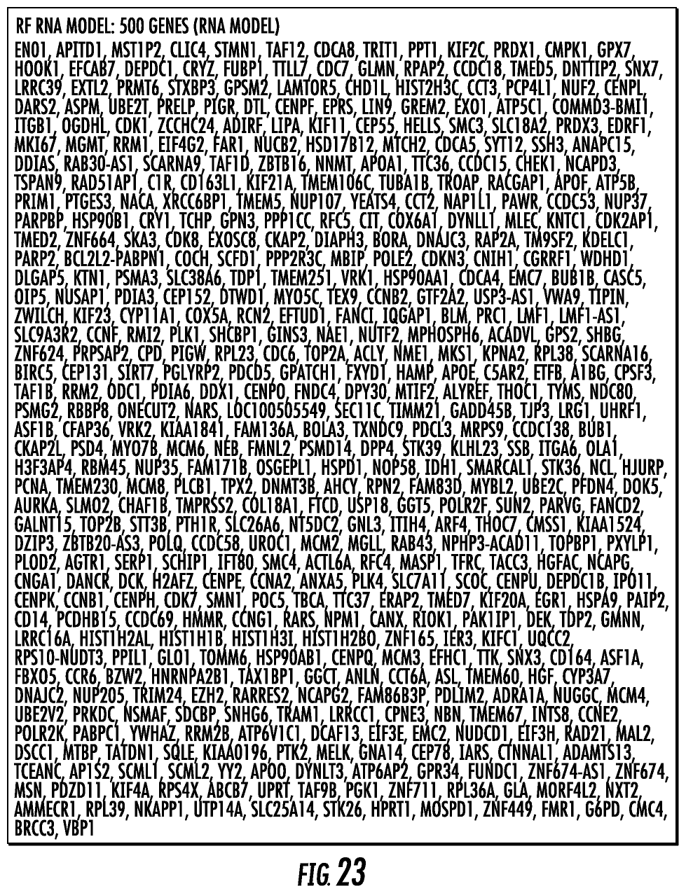

In some implementations, a random forest can be used to predict the subject's risk of recurrence of a liver cancer after liver transplantation model, and the gene expression data includes respective gene expression levels for a top-n differentially expressed genes, where n is an integer greater or equal to 10. Optionally, in the examples below, n is 500 (training and testing cohorts separate). Optionally, n is 50 (training and testing cohorts pooled). Although n=500 and n=50 are provided as examples, this disclosure contemplates that n can have other values including, but not limited to, 11, 12, 13, 14, 15, . . . 495, 496, 497, 498, 499, 500 or more. Optionally, in some implementations, n has a value greater than or equal to 50. Optionally, in some implementations, n has a value greater than or equal to 200. Optionally, in some implementations, n has a value between 50 and 500, and optionally between 200 and 500. In one example described herein, the top-500 differentially expressed genes are listed in FIG. 23. The attached sequence listing provides sequences for each of the genes identified in FIG. 23. This disclosure contemplates that each of the genes in FIG. 23 can have a sequence with 80, 85, 90, 95, etc. percent identity to its respective example sequence. In some implementations, the top-50 differentially expressed genes include HOOK1, EFCAB7, CDC7, NUF2, UBE2T, HELLS, RRM1, SYT12, KIF21A, RACGAP1, PRIM1, PTGES3, YEATS4, CCT2, PARPBP, PPP1CC, KNTC1, TMED2, CDKN3, DLGAP5, BUB1B, NUSAP1, CCNB2, KIF23, FANCI, PRC1, CDC6, TOP2A, KPNA2, NDC80, RBBP8, NARS, BUB1, TOPBP1, SMC4, NCAPG, CENPE, PLK4, CENPU, CENPQ, TTK, FBXO5, ANLN, MELK, DYNLT3, ZNF674, KIF4A, AMMECR1, ZNF449, and BRCC3. In some implementations, the top-500 differentially expressed genes include ENO1, APITD1, MSTIP2, CLIC4, STMN1, TAF12, CDCA8, TRIT1, PPT1, KIF2C, PRDX1, CMPK1, GPX7, HOOK1, EFCAB7, DEPDC1, CRYZ, FUBP1, TTLL7, CDC7, GLMN, RPAP2, CCDC18, TMED5, DNTTIP2, SNX7, LRRC39, EXTL2, PRMT6, STXBP3, GPSM2, LAMTOR5, CHDIL, HIST2H3C, CCT3, PCP4L1, NUF2, CENPL, DARS2, ASPM, UBE2T, PRELP, PIGR, DTL, CENPF, EPRS, LIN9, GREM2, EXO1, ATP5C1, COMMD3-BMI1, ITGB1, OGDHL, CDK1, ZCCHC24, ADIRF, LIPA, KIF11, CEP55, HELLS, SMC3, SLC18A2, PRDX3, EDRF1, MKI67, MGMT, RRM1, EIF4G2, FAR1, NUCB2, HSD17B12, MTCH2, CDCA5, SYT12, SSH3, ANAPC15, DDIAS, RAB30-AS1, SCARNA9, TAFID, ZBTB16, NNMT, APOA1, TTC36, CCDC15, CHEK1, NCAPD3, TSPAN9, RAD51AP1, CIR, CD163L1, KIF21A, TMEM106C, TUBA1B, TROAP, RACGAP1, APOF, ATP5B, PRIM1, PTGES3, NACA, XRCC6BP1, TMEM5, NUP107, YEATS4, CCT2, NAP1L1, PAWR, CCDC53, NUP37, PARPBP, HSP90B1, CRY1, TCHP, GPN3, PPP1CC, RFC5, CIT, COX6A1, DYNLL1, MLEC, KNTC1, CDK2AP1, TMED2, ZNF664, SKA3, CDK8, EXOSC8, CKAP2, DIAPH3, BORA, DNAJC3, RAP2A, TM9SF2, KDELC1, PARP2, BCL2L2-PABPN1, COCH, SCFD1, PPP2R3C, MBIP, POLE2, CDKN3, CNIH1, CGRRF1, WDHD1, DLGAP5, KTN1, PSMA3, SLC38A6, TDP1, TMEM251, VRK1, HSP90AA1, CDCA4, EMC7, BUB1B, CASC5, OIP5, NUSAP1, PDIA3, CEP152, DTWD1 ,MYO5C, TEX9, CCNB2, GTF2A2, USP3-AS1, VWA9, TIPIN, ZWILCH, KIF23, CYP11A1, COX5A, RCN2, EFTUD1, FANCI, IQGAP1, BLM, PRC1, LMF1, LMF1-AS1, SLC9A3R2, CCNF, RMI2, PLK1, SHCBP1, GINS3, NAE1, NUTF2, MPHOSPH6, ACADVL, GPS2, SHBG, ZNF624, PRPSAP2, CPD, PIGW, RPL23, CDC6, TOP2A, ACLY, NME1, MKS1, KPNA2, RPL38, SCARNA16, BIRC5, CEP131, SIRT7, ALYREF, THOC1, TYMS, NDC80, PSMG2, RBBP8, ONECUT2, NARS, LOC100505549, SEC11C, TIMM21, GADD45B, TJP3, LRG1, UHRF1, ASF1B, PGLYRP2, PDCD5, GPATCH1, FXYD1, HAMP, APOE, C5AR2, ETFB, A1BG, CPSF3, TAF1B, RRM2, ODC1, PDIA6, DDX1, CENPO, FNDC4, DPY30, MTIF2, CFAP36, VRK2, KIAA1841, FAM136A, BOLA3, TXNDC9, PDCL3, MRPS9, CCDC138, BUB1, CKAP2L, PSD4, MYO7B, MCM6, NEB, FMNL2, PSMD14, DPP4, STK39, KLHL23, SSB, ITGA6, OLA1, H3F3AP4, RBM45, NUP35, FAM171B, OSGEPL1, HSPD1, NOP58, IDH1, SMARCAL1, STK36, NCL, HJURP, PCNA, TMEM230, MCM8, PLCB1, TPX2, DNMT3B, AHCY, RPN2, FAM83D, MYBL2, UBE2C, PFDN4, DOK5, AURKA, SLMO2, CHAF1B, TMPRSS2, COL18A1, FTCD, USP18, GGT5, POLR2F, SUN2, PARVG, FANCD2, GALNT15, TOP2B, STT3B, PTH1R, SLC26A6, NT5DC2, GNL3, ITIH4, ARF4, THOC7, CMSS1, KIAA1524, DZIP3, ZBTB20-AS3, POLQ, CCDC58, UROC1, MCM2, MGLL, RAB43, NPHP3-ACAD11, TOPBP1, PXYLP1, PLOD2, AGTR1, SERP1, SCHIP1, IFT80, SMC4, ACTL6A, RFC4, MASP1, TFRC, TACC3, HGFAC, NCAPG, CNGA1, DANCR, DCK, H2AFZ, CENPE, CCNA2, ANXA5, PLK4, SLC7A11, SCOC, CENPU, DEPDC1B, IPO11, CENPK, CCNB1, CENPH, CDK7, SMN1, POC5, TBCA, TTC37, ERAP2, TMED7, KIF20A, EGR1, HSPA9, PAIP2, CD14, PCDHB15, CCDC69, HMMR, CCNG1, RARS, NPM1, CANX, RIOK1, PAK1IP1, DEK, TDP2, GMNN, LRRC16A, HIST1H2AL, HIST1H1B, HIST1H3I, HIST1H2BO, ZNF165, IER3, KIFC1, UQCC2, RPS10-NUDT3, PPIL1, GLO1, TOMM6, HSP90AB1, CENPQ, MCM3, EFHC1, TTK, SNX3, CD164, ASF1A, FBX05, CCR6, BZW2, HNRNPA2B1, TAX1BP1, GGCT, ANLN, CCT6A, ASL, TMEM60, HGF, CYP3A7, DNAJC2, NUP205, TRIM24, EZH2, RARRES2, NCAPG2, FAM86B3P, PDLIM2, ADRA1A, NUGGC, MCM4, UBE2V2, PRKDC, NSMAF, SDCBP, SNHG6, TRAM1, LRRCC1, CPNE3, NBN, TMEM67, INTS8, CCNE2, POLR2K, PABPC1, YWHAZ, RRM2B, ATP6V1C1, DCAF13, EIF3E, EMC2, NUDCD1, EIF3H, RAD21, MAL2, DSCC1, MTBP, TATDN1, SQLE, KIAA0196, PTK2, MELK, GNA14, CEP78, IARS, CTNNAL1, ADAMTS13, TCEANC, APIS2, SCML1, SCML2, YY2, APOO, DYNLT3, ATP6AP2, GPR34, FUNDC1, ZNF674-AS1, ZNF674, MSN, PDZD11, KIF4A, RPS4X, ABCB7, UPRT, TAF9B, PGK1, ZNF711, RPL36A, GLA, MORF4L2, NXT2, AMMECR1, RPL39, NKAPP1, UTP14A, SLC25A14, STK26, HPRT1, MOSPD1, ZNF449, FMR1, G6PD, CMC4, BRCC3, and VBP1.

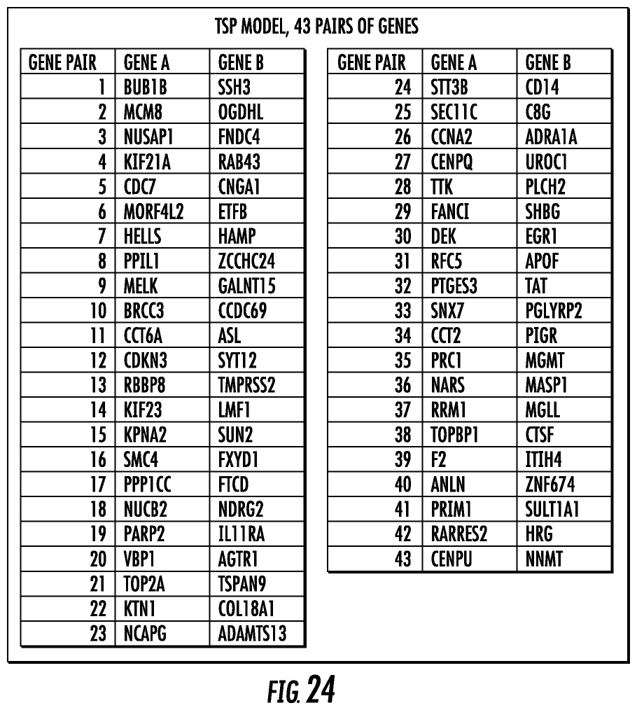

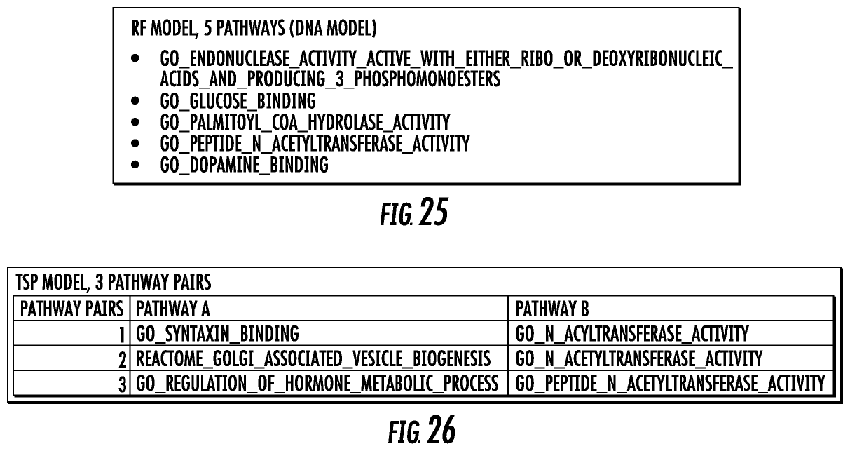

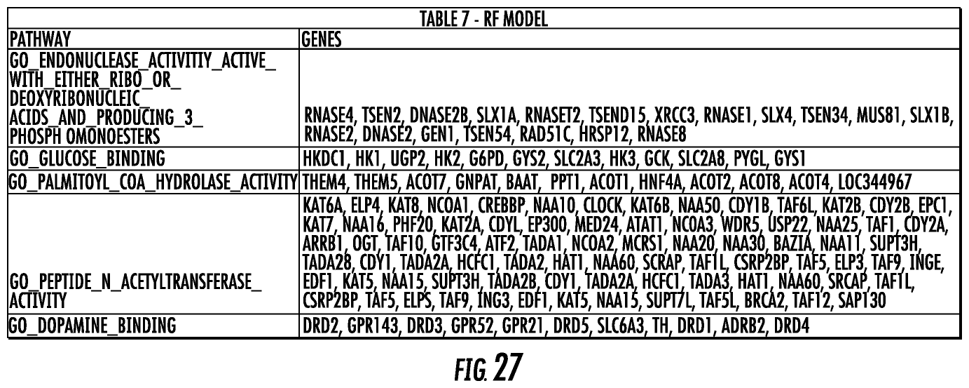

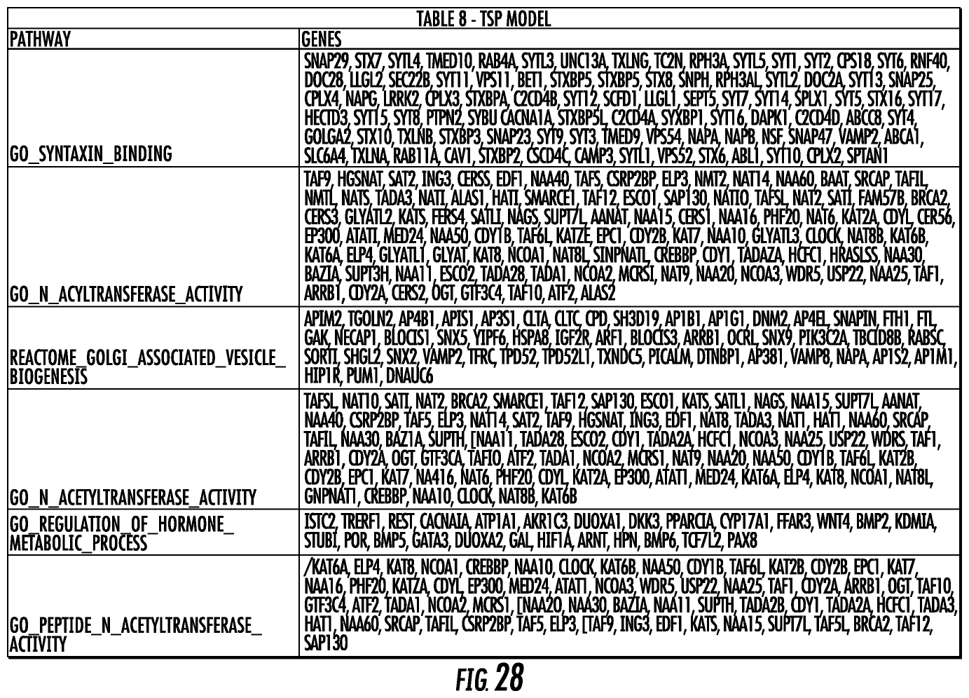

In some implementations, a k-TSP model can be used to predict the subject's risk of recurrence of a liver cancer after liver transplantation model, and the gene expression data includes respective gene expression levels for a top-q pairs of differentially expressed genes, where q is an integer greater or equal to 10. Optionally, in the examples below, q is 23 (training and testing cohorts separate). Optionally, q is 47 (training and testing cohorts pooled). Although q=23 and q=47 are provided as examples, this disclosure contemplates that n can have other values including, but not limited to, 11, 12, 13, 14, 15, . . . 44, 45, 46, 47 or more. In one example described herein, the top-43 pairs of differentially expressed genes are listed in the table shown in FIG. 24. The attached sequence listing provides sequences for each of the genes identified in FIG. 24. This disclosure contemplates that each of the genes in FIG. 24 can have a sequence with 80, 85, 90, 95, etc. percent identity to its respective example sequence.

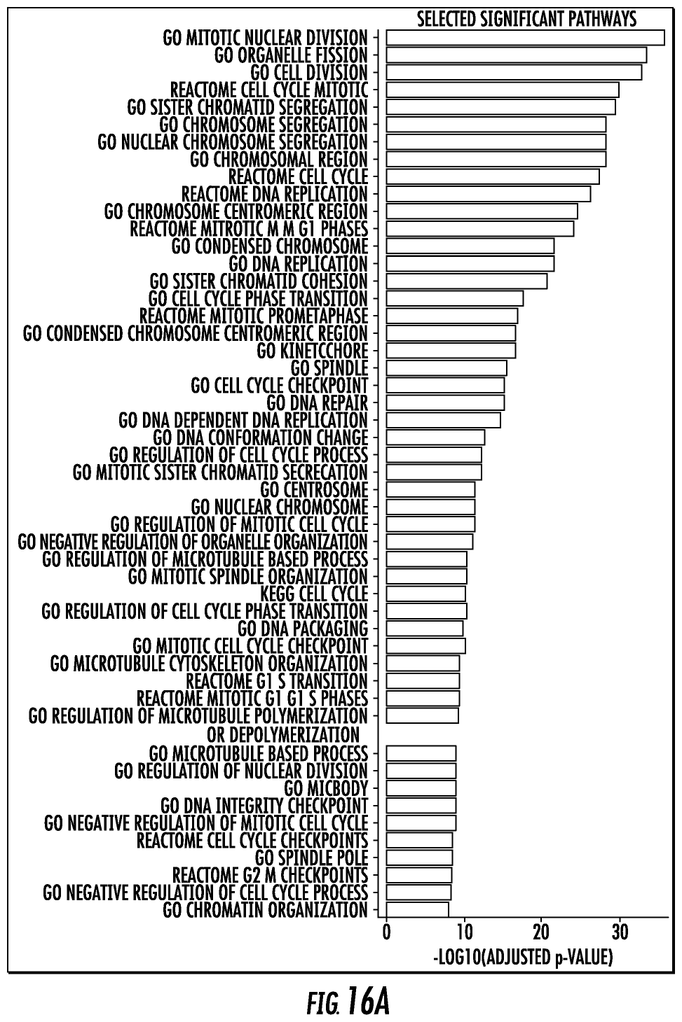

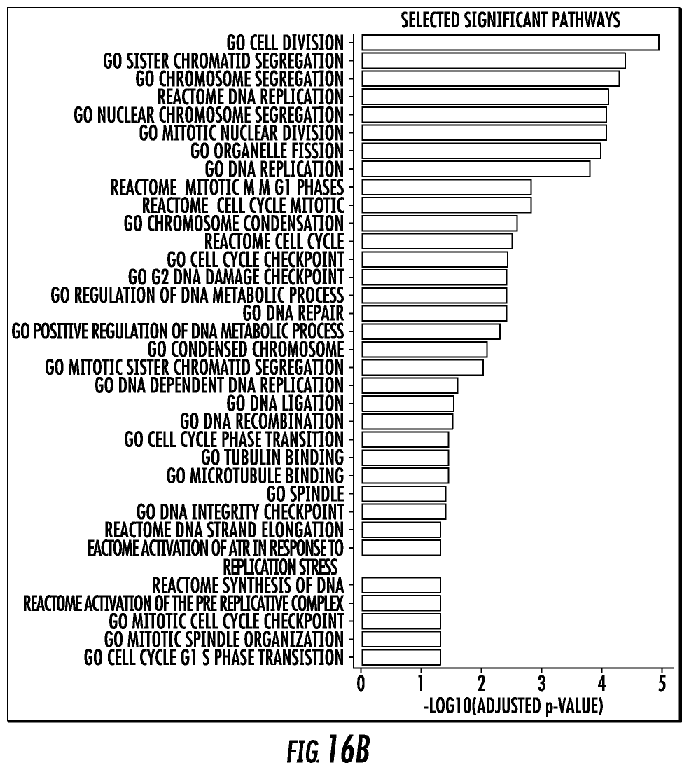

Additionally, in some implementations described herein, the input 110 includes data from an exome analysis. In other words, the input 110 can include data from both transcriptome and exome analyses. For example, the input 110 to the machine learning model 100 can further include mutation data related to the liver tissue sample. This disclosure contemplates that mutation data can be obtained using techniques known in the art. For example, mutation data can be obtained by sampling liver tissue, extracting Deoxyribonucleic acid (DNA) from the sample, sequencing the DNA, and identifying somatic mutations. Somatic mutations can be identified based on a comparison of the liver tissue sample DNA sequences to a control set of DNA sequences derived from a control subject or population that either has no cancer or no cancer recurrence. In some implementations, the mutation data includes a number of somatic mutations present in the liver tissue sample. In some implementations, the mutation data includes a number of somatic mutations present in a top-m mutation pathways, where m is an integer greater or equal to 5. Optionally, m is 5. Although m=5 is provided as an example, this disclosure contemplates that m can have other values including, but not limited to, 6, 7, 8, 9, . . . 26, 27, 28, 29, 30 or more. FIGS. 16A and 16B illustrate example ranking of pathways impacted by expression alterations according to examples described herein. Additionally, FIG. 25 illustrates the top-5 mutation pathways impacted by expression alterations for random forest modelling according to one example. The genes associated with the top-5 mutation pathways of FIG. 25 are listed in the Table 7 shown in FIG. 27. The attached sequence listing provides sequences for each of the genes identified in Table 7. This disclosure contemplates that each of the genes in Table 7 can have a sequence with 80, 85, 90, 95, etc. percent identity to its respective example sequence. Alternatively or additionally, in some implementations, the mutation data includes a number of somatic mutations present in a top-r mutation pathways pairs, where r is an integer greater or equal to 3. Optionally, ris 3. Although r=3 is provided as an example, this disclosure contemplates that r can have other values including, but not limited to, 4, 5, 6, 7, 8, 9, . . . 26, 27, 28, 29, 30 or more. Additionally, FIG. 26 includes a table that illustrates the top-3 mutation pathway pairs impacted by expression alterations for k-TSP modelling according to one example. The genes associated with the top-3 mutation pathways pairs of FIG. 26 are listed in the Table 8 shown in FIG. 28. The attached sequence listing provides sequences for each of the genes identified in Table 8. This disclosure contemplates that each of the genes in Table 8 can have a sequence with 80, 85, 90, 95, etc. percent identity to its respective example sequence. In one example described herein, a random forest model is used to predict the subject's risk of recurrence of a liver cancer after liver transplantation based on features from the transcriptome and exome analyses. For example, FIGS. 4C, 4D, 4F, 5C, 5D, and 5F illustrate analysis of a random forest prediction model according to examples described herein. This disclosure contemplates that other machine learning models such as an SVM, a logistic regression model, or a k-TSP model can be used to predict the subject's risk of recurrence of a liver cancer after liver transplantation based on features from the transcriptome and exome analyses. For example, FIGS. 11C, 11D, 12C, and 12D illustrate analysis of a k-TSP prediction model according to examples described herein.

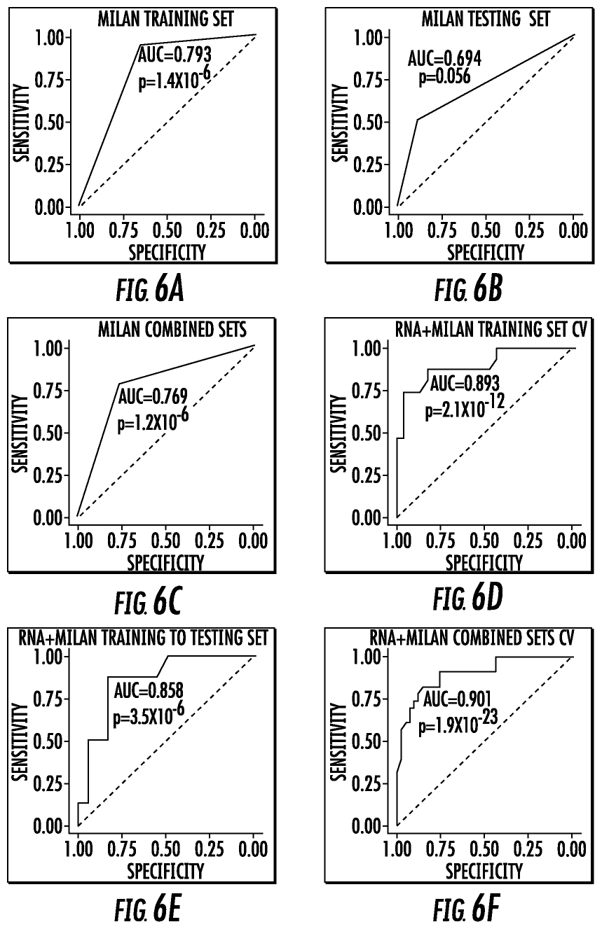

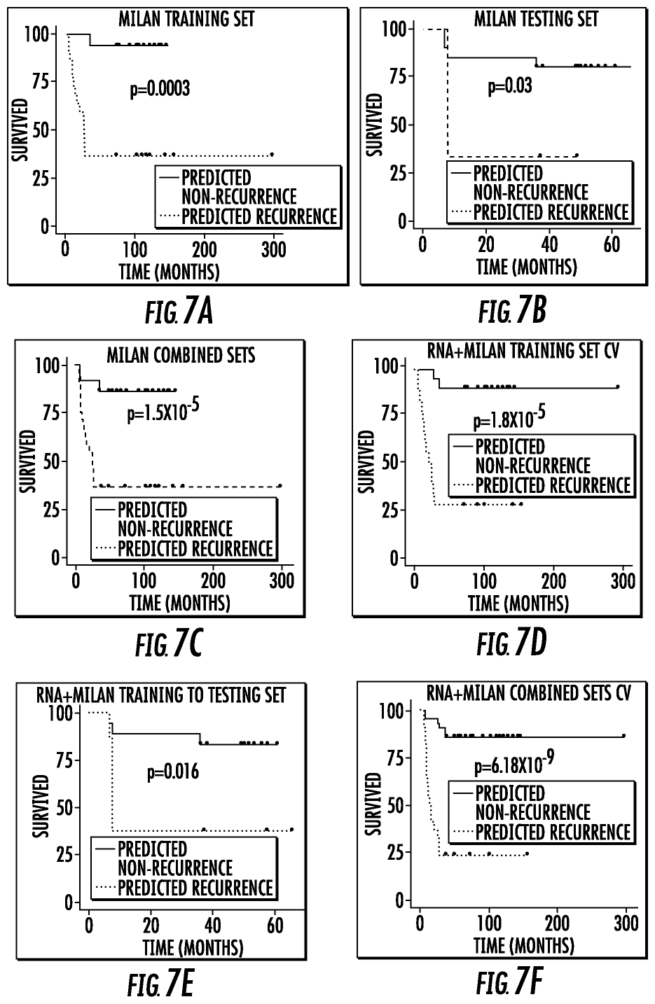

Additionally, in some implementations described herein, the input 110 includes data from an imaging analysis. In other words, the input 110 can include data from both transcriptome and imaging analyses. This disclosure contemplates that imaging modalities for detecting and characterizing liver tumors include, but are not limited to, ultrasound, computed tomography (CT), magnetic resonance imaging (MRI), and positron emission tomography (PET). It should be understood that ultrasound, CT, MRI, and PET are provided only as examples and that other imaging modalities may be used. For example, the input 110 to the machine learning model 100 can include one or more radiology-based parameters related to the liver cancer. In some implementations, a radiology-based parameter is based on a number of tumor nodules associated with the liver cancer. Alternatively or additionally, a radiology-based parameter is based on a size of tumor nodules associated with the liver cancer. This disclosure contemplates that a radiology-based parameter can be based on other characteristics of the liver cancer. In some implementations, the radiology-based parameter can be based on a combination of characteristics of the liver cancer (e.g., number and size of nodules). Optionally, the radiology-based parameter is Milan criteria, which are a set of criteria used to assess a subject's candidacy for liver transplantation. The Milan criteria are known in the art and therefore not described in further detail herein. Although Milan criteria are provided as an example radiology-based parameter, this disclosure contemplates using other metrics (e.g., the extended Toronto criteria) to characterize the liver cancer. In one example described herein, a k-TSP model is used to predict the subject's risk of recurrence of a liver cancer after liver transplantation based on features from the transcriptome and imaging analyses. For example, FIGS. 6D, 6E, 6F, 7D, 7E, and 7F illustrate analysis of a k-TSP prediction model according to examples described herein. This disclosure contemplates that other machine learning models such as an SVM, a random forest model, or a logistic regression model can be used to predict the subject's risk of recurrence of a liver cancer after liver transplantation based on features from the transcriptome and imaging analyses.

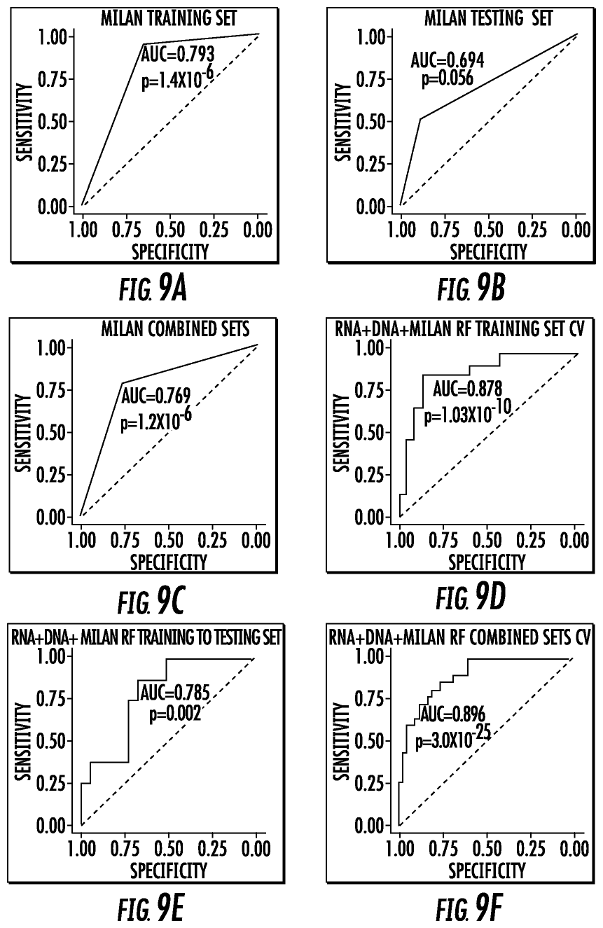

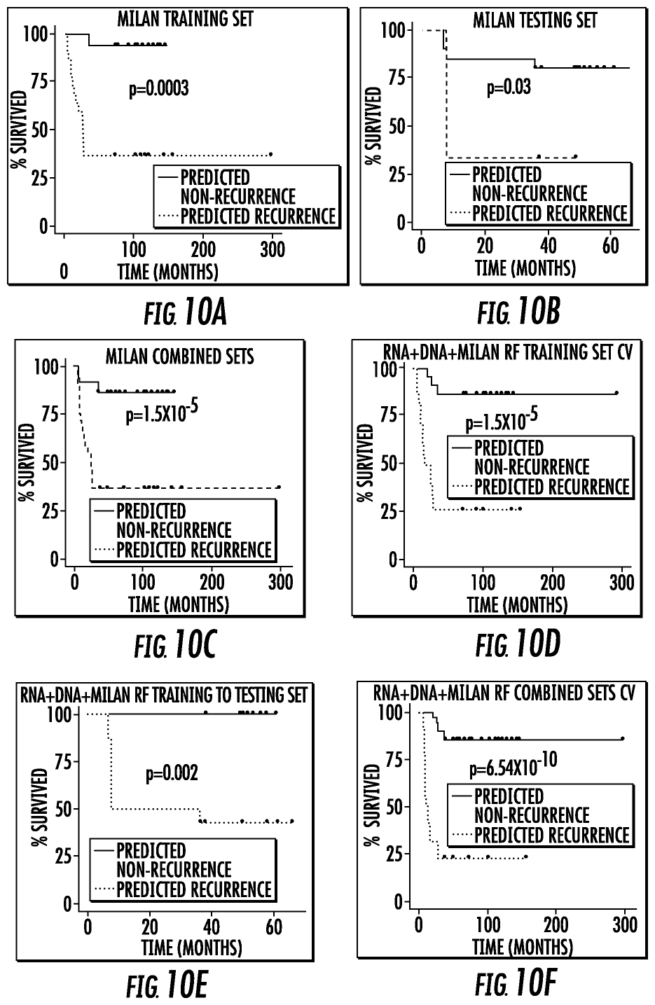

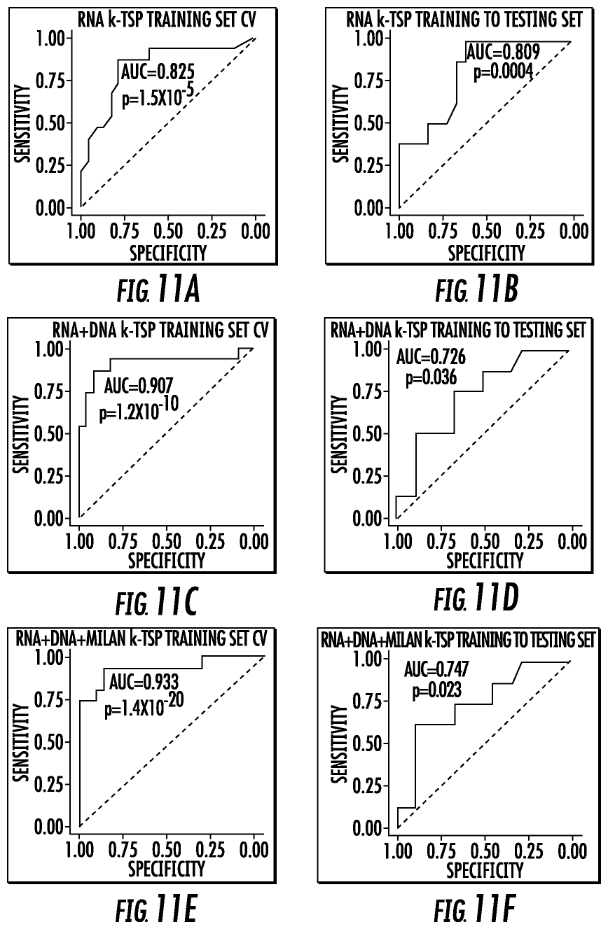

Alternatively, in some implementations described herein, the input 110 includes data from transcriptome, exome, and imaging analyses. In other words, the input 110 to the machine learning model 100 can include gene expression data, mutation data, and radiology-based parameters related to the liver cancer. In one example described herein, a random forest model is used to predict the subject's risk of recurrence of a liver cancer after liver transplantation based on features from the transcriptome, exome, and imaging analyses. For example, FIGS. 9D, 9E, 9F, 10D, 10E, and 10F illustrate analysis of a random forest prediction model according to examples described herein. This disclosure contemplates that other machine learning models such as an SVM, a k-TSP model, or a logistic regression model is used to predict the subject's risk of recurrence of a liver cancer after liver transplantation based on features from the transcriptome, exome, and imaging analyses. For example, FIGS. 11E, 11F, 12E, and 12F illustrate analysis of a k-TSP prediction model according to examples described herein.

It should be appreciated that the logical operations described herein with respect to the various figures may be implemented (1) as a sequence of computer implemented acts or program modules (i.e., software) running on a computing device (e.g., the computing device described in FIG. 2), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 2, an example computing device 200 upon which the methods described herein may be implemented is illustrated. It should be understood that the example computing device 200 is only one example of a suitable computing environment upon which the methods described herein may be implemented. Optionally, the computing device 200 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 200 typically includes at least one processing unit 206 and system memory 204. Depending on the exact configuration and type of computing device, system memory 204 may be volatile (such as random access memory (RAM)), non-volatile (such as read-only memory (ROM), flash memory, etc.), or some combination of the two. This most basic configuration is illustrated in FIG. 2 by dashed line 202. The processing unit 206 may be a standard programmable processor that performs arithmetic and logic operations necessary for operation of the computing device 200. The computing device 200 may also include a bus or other communication mechanism for communicating information among various components of the computing device 200.

Computing device 200 may have additional features/functionality. For example, computing device 200 may include additional storage such as removable storage 208 and non-removable storage 210 including, but not limited to, magnetic or optical disks or tapes. Computing device 200 may also contain network connection(s) 216 that allow the device to communicate with other devices. Computing device 200 may also have input device(s) 214 such as a keyboard, mouse, touch screen, etc. Output device(s) 212 such as a display, speakers, printer, etc. may also be included. The additional devices may be connected to the bus in order to facilitate communication of data among the components of the computing device 200. All these devices are well known in the art and need not be discussed at length here.

The processing unit 206 may be configured to execute program code encoded in tangible, computer-readable media. Tangible, computer-readable media refers to any media that is capable of providing data that causes the computing device 200 (i.e., a machine) to operate in a particular fashion. Various computer-readable media may be utilized to provide instructions to the processing unit 206 for execution. Example tangible, computer-readable media may include, but is not limited to, volatile media, non-volatile media, removable media and non-removable media implemented in any method or technology for storage of information such as computer readable instructions, data structures, program modules or other data. System memory 204, removable storage 208, and non-removable storage 210 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 206 may execute program code stored in the system memory 204. For example, the bus may carry data to the system memory 204, from which the processing unit 206 receives and executes instructions. The data received by the system memory 204 may optionally be stored on the removable storage 208 or the non-removable storage 210 before or after execution by the processing unit 206.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

### Example Methods

Provided herein are methods of predicting a risk of recurrence of the liver cancer in the subject after liver transplantation. For example, the method can include receiving gene expression data related to a liver tissue sample for a subject having a liver cancer. This disclosure contemplates that the gene expression data can be received by a computing device such as the computing device 200 shown in FIG. 2. The method can also include inputting the gene expression data into a trained machine learning model such as the machine learning model 100 shown in FIG. 1. Gene expression data is described above and therefore not described in further detail here. The method can further include predicting, using the trained machine learning model, a risk of recurrence of the liver cancer in the subject after liver transplantation. In these implementations, the subject's risk of recurrence of a liver cancer after liver transplantation is predicted based on features from the transcriptome analysis alone.

Optionally, in some implementations, the method can further include receiving mutation data related to the liver tissue sample; and inputting the mutation data into the trained machine learning model. Mutation data is described above and therefore not described in further detail here. In these implementations, the subject's risk of recurrence of a liver cancer after liver transplantation is predicted features on data from both the transcriptome and exome analyses.

Optionally, in some implementations, the method can further include receiving a radiology-based parameter related to the liver cancer; and inputting the radiology-based parameter into the trained machine learning model. Radiology-based parameters are described above and therefore not described in further detail here. In these implementations, the subject's risk of recurrence of a liver cancer after liver transplantation is predicted based on features from both the transcriptome and imaging analyses.

Optionally, in some implementations, the method can further include receiving mutation data related to the liver tissue sample and a radiology-based parameter related to the liver cancer; and inputting the mutation data and the radiology-based parameter into the trained machine learning model. In these implementations, the subject's risk of recurrence of a liver cancer after liver transplantation is predicted based on features from all three of the transcriptome, exome, and imaging analyses.

Optionally, in some implementations, the method can further include providing a treatment recommendation based on the prediction. For example, the treatment recommendation is optionally to perform a liver transplant procedure on the subject. It should be understood a subject with a relatively lower risk of cancer recurrence is a better candidate for liver transplantation than a subject with a relatively higher risk of cancer recurrence. In other words, as described herein, it is possible that a liver cancer such as HCC recurs in a subject even after liver transplantation. The machine learning-based systems and methods described herein are capable of predicting the risk of recurrence of liver cancer, and such information can be used to provide a treatment recommendation such as selecting a suitable candidate for liver transplantation.

Optionally, in some implementations, the method can further include performing a liver transplant procedure on a subject. For example, the liver transplant procedure can be performed on a subject for which the machine learning-based systems and methods described herein have predicted a relatively low risk of cancer recurrence.

It should also be understood that the foregoing relates to preferred embodiments of the present invention and that numerous changes may be made therein without departing from the scope of the invention. The invention is further illustrated by the following examples, which are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the present invention and/or the scope of the appended claims. All patents, patent applications, and publications referenced herein are incorporated by reference in their entirety for all purposes.

### EXAMPLES

### Example 1

### Methods

All the human samples in the experiment were obtained in accordance with the guidelines approved by the Institutional Review Board of the University of Pittsburgh. All the methods were carried out in accordance with relevant guidelines and regulations. Informed-consent exemptions were obtained from University of Pittsburgh Institutional Review Board.

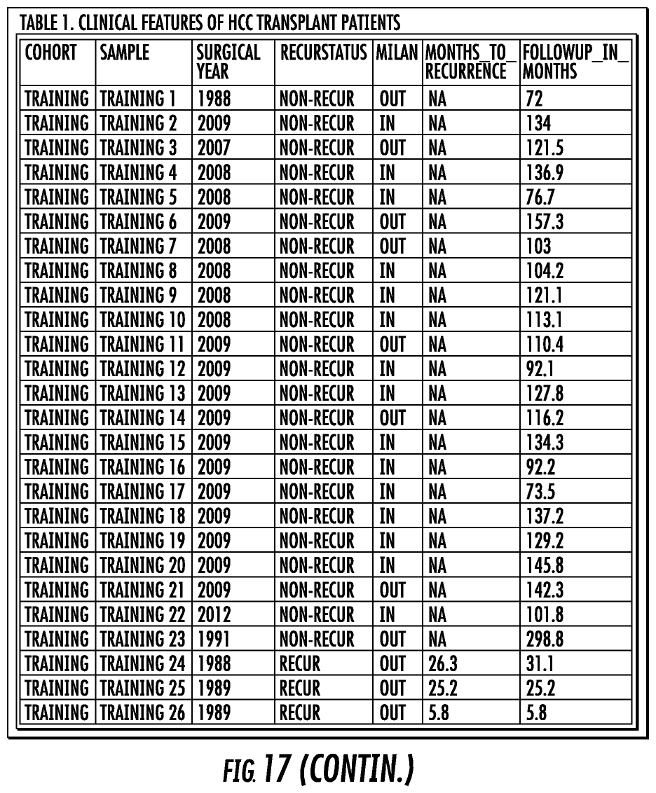

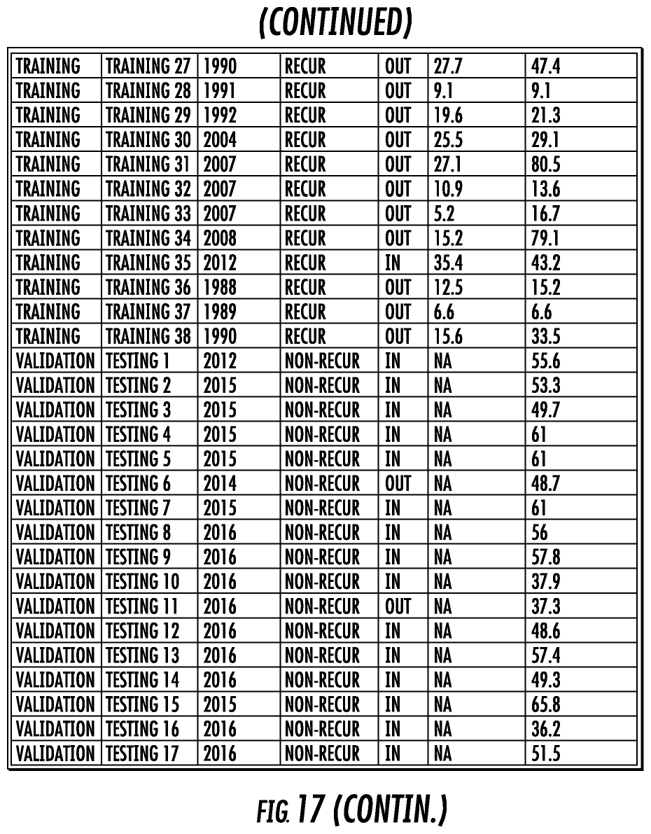

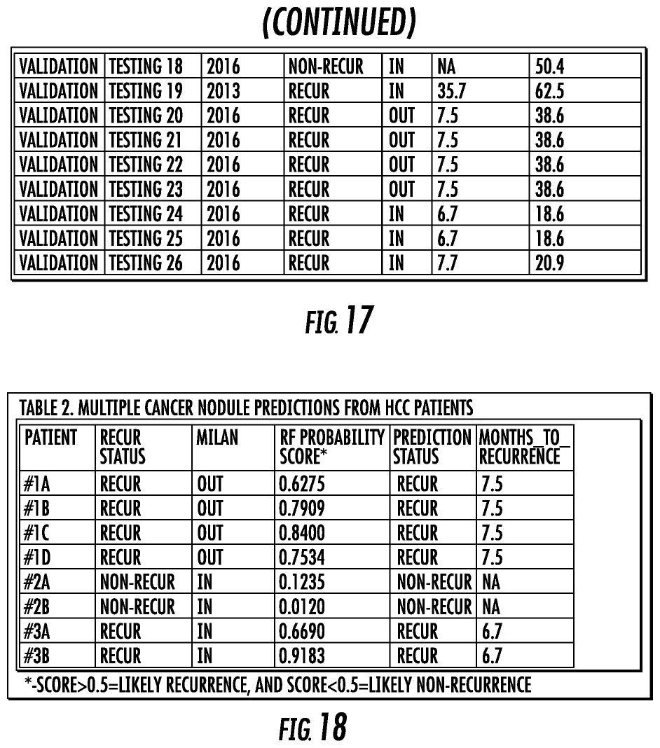

Tissue samples. The 128 tissue specimens in the study were obtained from the University of Pittsburgh Medical Center archived tissue deposit center in compliance with institutional regulatory guidelines (See FIG. 17, Table 1). Cancer tissues were identified through Hematoxylin and Eosin staining. The position of the cancer in the slide was matched with the tissue block, and circled. The identified positions were then used to obtain needle cores from the cancer tissues. Non-liver and benign tissues away from the cancer were used as matched normal control. The sample size was estimated by power analysis and availability of the clinical specimens. The processes and protocols followed the guidelines approved by the Institutional Review Board of University of Pittsburgh. All cancer samples were obtained through needle coring of the paraffin tissue blocks.

Transcriptome sequencing. Paraffin was removed by incubating the tissue cores with xylene overnight. The RNA extraction and the transcriptome sequencing procedures were described previously (10-17). Briefly, total RNA was extracted from the tissue cores using TRIzol methods. DNase1 was used to degrade DNA, and a RIBO-Zero™ Magnetic Kit (Epicentre, Madison, WI) was used to remove ribosomal RNA from the samples. RNA was reverse transcribed to cDNA, and a TruSeq·8 RNA Sample Prep Kit v2 (Illumina, Inc. San Diego, CA) was used for library preparation. The procedure was guided by the manufacturer's manual. The quality of the transcriptome library was analyzed with qPCR using Illumina sequencing primers and quantified in an Agilent 2000 Bioanalyzer. The sequencing procedure followed the manual for paired-end sequencing with 200 cycles as specified for the HiSeq 2500 or with 300 cycles as specified for the NextSeq550 platform by Illumina.

Exome sequencing. Illumina TruSeq DNA Exome prep kit was used to prepare the exome library. Briefly, the extracted DNA (100 ng) was fragmented in Covaris sonicator to 200 bp length. This was followed by ends repairing, adenylation of 3′ end, and adapters ligation. After the clean-up by magnetic beads, the DNA fragments were PCR amplified for 8 cycles of 98° C. 20 seconds, 60° C. for 20 seconds and 72° C. for 30 seconds. The amplified DNA was applied to hybridize the probes, and the hybridized probes were captured by Streptavidin magnetic beads. After repeating the probe hybridization and probe capturing, the enriched DNA fragments were amplified for 8 cycles of 98° C. for 10 seconds, 60° C. for 35 seconds, and 72° C. for 30 seconds. The libraries were then assessed for quality and quantity in an Agilent 2000 Bioanalyzer. The sequencing procedure followed the manual for paired-end sequencing with 200 cycles as specified for the HiSeq 2500 or with 300 cycles as specified for the NextSeq550 platform by Illumina.

Bioinformatics analysis for transcriptome sequencing data. The sequencing quality control was first performed on RNA-seq data through FastQC (https://www.bioinformatics.babraham.ac.uk/projects/fastqc/). Adapter sequences and low quality reads were trimmed out by Trimmomatic (18). After pre-processing, surviving reads were aligned to human reference genome hg19 by aligner Hisat2 (19). Gene fragments per kilobase per million reads (FPKM) were quantified by Cufflinks (20). All the pipelines were run by default parameters.

Bioinformatics analysis for whole-exome sequencing data. DNA specimens from paired data (tumor and benign tissue for the same patient) were collected for whole exome sequencing (WES). Similar as RNA-seq data, each WES data first went through the pipeline of quality control (https://www.bioinformatics.babraham.ac.uk/projects/fastqc/) and filtering (18). Reads that passed the quality control were then aligned to human reference genome hg19 by Burrows-Wheeler Aligner mem function (21). Tool Picard (http://broadinstitute.github.io/picard/) was then applied to sort, index and mark duplicates on the aligned reads. Genome Analysis Toolkit (22) analysis pipeline was then employed to perform the realignment and mutation calling. Eventually, paired-samples (tumor and normal) were matched to call somatic mutation by GATK Mutect2 (22). All the pipelines were run by default parameters.

Prediction model on transcriptome expression profiles. Genome-wide gene expression profiles were quantified across all the tumor cases. FPKM values were first log2 scaled. Several machine learning algorithms were applied on the transcriptome expression data: support vector machine (SVM) (23), random forest (RF) (24, 25), linear discriminant analysis (LDA) (26), logistic regression (27) and k-top scoring pairs (k-TSP) (28). Quantile normalization across the training and testing cohorts was applied to correct the batch effect for the first four algorithms, while k-TSP is a non-parametric method where quantile normalization is not required. For all these methods, leave-one-out cross-validation (LOOCV) was performed on the training cohort to evaluate the prediction algorithms and select the best parameters (the best top number of genes or paired-genes). Then the best algorithm was applied to the whole training cohort to train a model and apply to the testing cohort. Eventually, the training and testing cohorts were pooled together to generate the best model for the prediction of recurrence of a new case. All the biostatistical analyses were performed by R programming and available R packages: ‘randomForest’, ‘MASS’, ‘e1071’ and ‘switchBox’ (29).

Prediction model integrating transcriptome expression and gene mutation. All the machine learning algorithms applied to transcriptome analysis were used to integrate both RNA and DNA data. At the RNA level, gene expressions were used as features, which is similar to the model only working on transcriptome expression data. At the DNA level, somatic mutations were called on each tumor-normal pair individually. Known Gene Ontology (GO) and Kyoto Encyclopedia of Genes and Genomes (KEGG) pathways with defined functional gene sets were collected from the public database (30, 31). Total number of genes with somatic mutations was then calculated for each functional pathway and used as DNA-level features.

For the machine learning models RF, SVM, LDA and logistic regression, both transcriptome expression at RNA-level and pathway mutation at DNA-level were employed as prediction features. Random forest regression prediction was used to predict a probability score for recurrence. The score ranges from 0 to 1, where score>0.5 represents recurrence and score<0.5 means non-recurrence prediction. For k-TSP model, it was applied to transcriptome expression and gene mutation profiles individually. To combine the two omics datasets, scores calculated from the transcriptome expression data and scores from the gene mutation data were weighted and summed for the prediction. The final score ranges from −1 to 1, where a positive score represents recurrence and a negative score means non-recurrence for binary prediction. Similar to the model only involving transcriptome expression data, the model integrating both the RNA and DNA was first applied to the training cohort. The best parameters selected by LOOCV were used as the final model for the training cohort and then applied to the testing cohort for evaluation. In the final stage, both cohorts were pooled to provide a final prediction model on leave-one-out cross-validation. All the biostatistical analyses were performed by R programming and available R packages (29).

Prediction model integrating transcriptome expression, gene mutation and Milan score. Similar to transcriptome expression and gene mutation integration, multiple machine learning models were employed to integrate RNA expression, DNA mutation, and Milan score. For k-TSP model, it assigned a weight to the RNA score, DNA score and Milan score (−1 for ‘in’ and 1 for ‘out’). The final prediction score is the sum of all three weighted scores. For RF, SVM, LDA and logistic regression, the following were employed as features contributing to prediction: gene expression, pathway mutation, and Milan score. RF generated a probability score ranging from 0 to 1, where a score higher than 0.5 indicates recurrence, and a score less than 0.5 predicts nonrecurrence.

Downstream functional pathway analysis. When combining the training and testing data, the top 500 differentially expressed genes (DEGs) were selected by the ranking of p-values. These genes were then used for functional pathway analysis. Four pathway databases were collected for the enrichment test: Gene Ontology (GO) (30), Kyoto Encyclopedia of Genes and Genomes (KEGG) (31), Reactome (32) and BioCarta (33). Top significant enriched pathways were selected by FDR=5%. Genes involved in selected pathways were used for network analysis. Clustering heatmap, pathway barplot and network figure were generated by R programming (package ComplexHeatmap (34) and ggplot (35) and Cytoscape software (36).

Statistical analysis. All the statistical analyses were performed by R programming. The receiver operating characteristic (ROC) curves and Kaplan-Meier analyses were analyzed and plotted by R/Bioconductor packages survival (https://CRAN.R-project.org/package=survival), PROC (37), ggfortify (38) and GGally (https://CRAN.R-project.org/package=GGally).

### Example 2

### Pre-Determination of Training Cohort and Testing Cohort

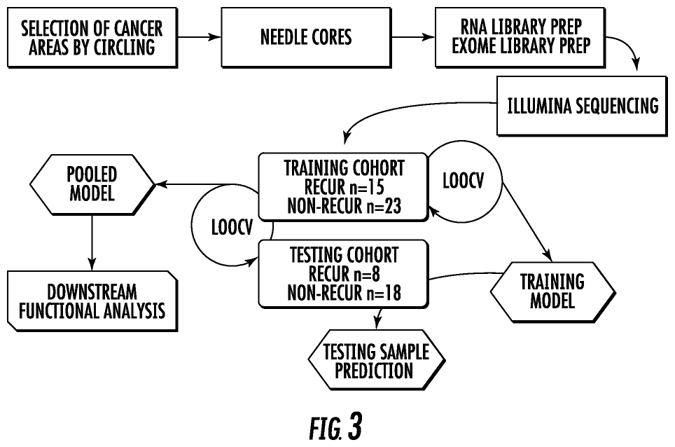

In previous studies, it was shown that the alterations of genome and gene expression occur in human hepatocellular carcinoma and are associated with the aggressiveness of the cancers (39, 40). However, it is unclear whether these changes contain predictive values for the clinical prognosis of HCC patients undergoing liver transplants. To examine whether the alterations of gene expressions and genome in the HCC are predictive of the cancer recurrence of HCC in the liver transplant patients, two cohorts based on the surgical timeline were constructed for transcriptome and exome sequencing analyses. The training cohort (38 cases) included HCC samples obtained from patients who had liver transplants from 1988 to the first half of 2012, while the testing cohort (26 cases) included HCC samples from the second half of 2012 to 2019. The results of the transcriptome and exome analyses of the training cohort were combined to develop a classification algorithm as a training set (FIG. 3). The algorithm was then applied to predict the clinical outcomes of the cases from the testing set (second cohort).

### Example 3

### Transcriptome Sequencing to Predict HCC Outcomes

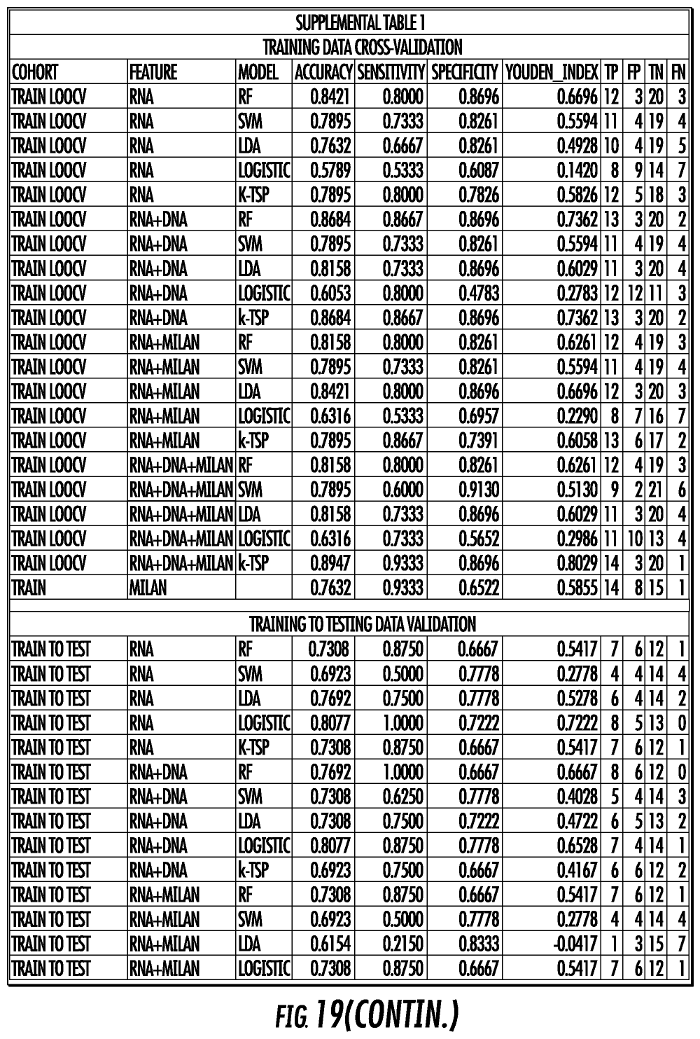

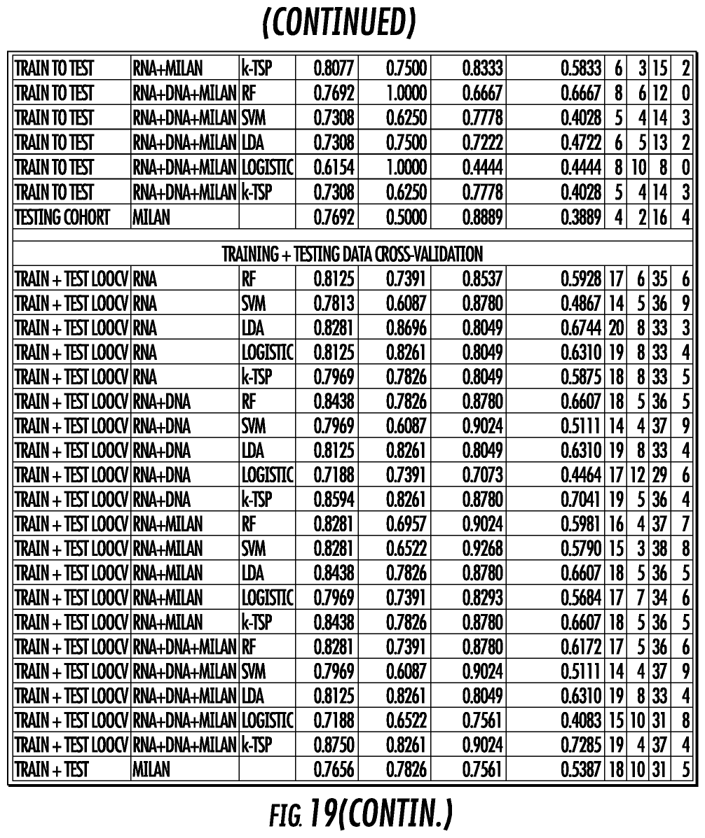

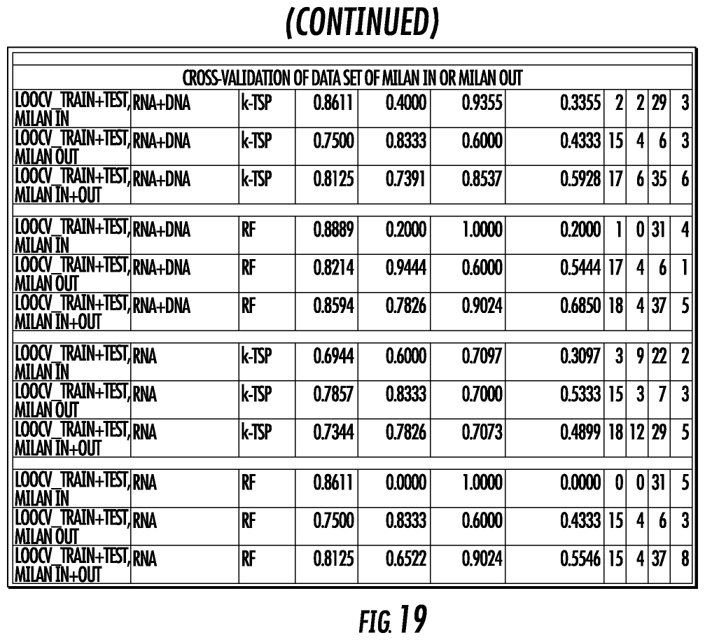

The transcriptome analysis was performed using Random Forest (RF) (24, 25) model where all the genes were ranked based on their differential expression between recurrence and nonrecurrence samples. The top 10 differentially expressed genes were first used to predict the recurrence status of the cases in the training set using the leave-one-out cross-validation (LOOCV) method. Subsequently, top 20, 30, 40, 50, 100, 200, 500, or 1000 differentially expressed genes were added to train the model and to examine whether addition of genes improved the results. The final model was selected based on the best Youden Index (sensitivity +specificity −1). As shown in FIG. 4A, 500 differentially expressed genes were found to produce the best results in predicting the cancer recurrence for HCC patients. The Receiver Operating Characteristic (ROC) curve yielded an area under the curve (AUC) of 0.87 and a p-value of 2.8×10⁻⁹. The LOOCV model based on 500 genes produced 84.2% accuracy, with a sensitivity of 80% and specificity of 87% (FIG. 19, Supplemental Table 1). When this algorithm was applied to the testing cohort, the AUC of the ROC was 0.806 and a p-value of 0.00049 (FIG. 4B). The accuracy was 73.1%, with 87.5% sensitivity and 66.7% specificity (FIG. 19, Supplemental Table 1).

### Example 4

### Mutation Pathways Analysis to Enhance Prediction of HCC Outcomes

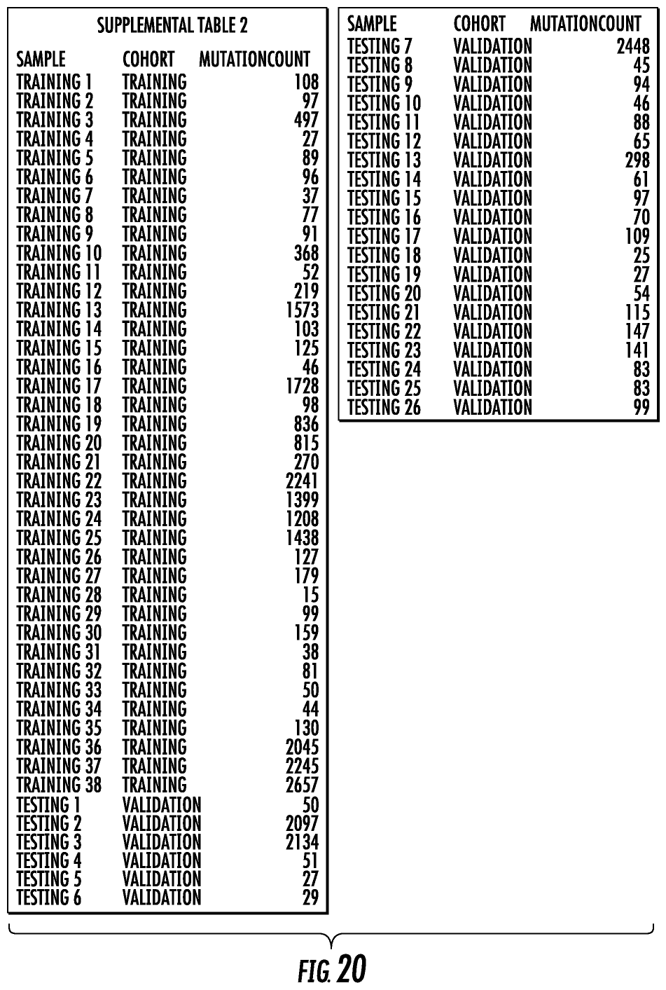

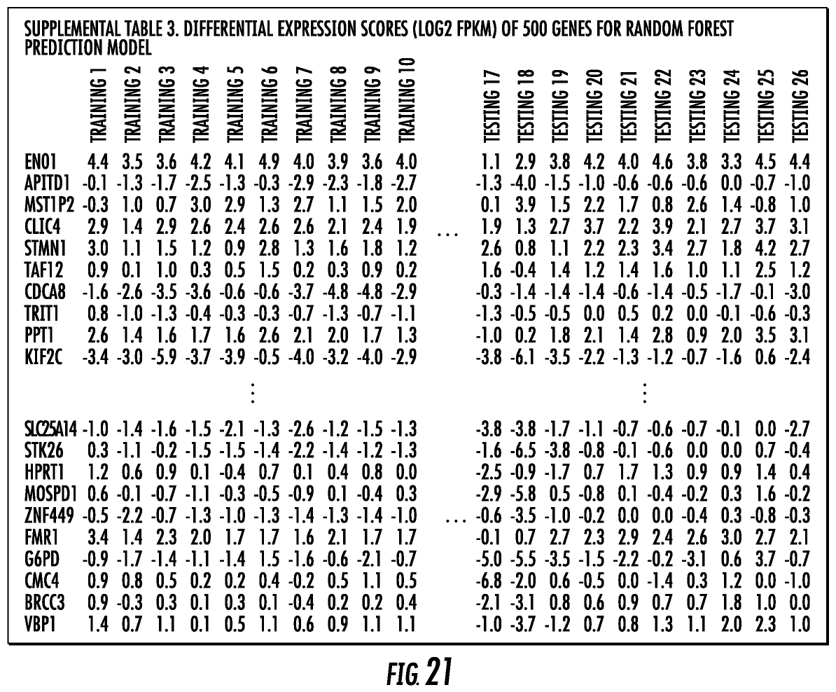

To examine whether genome mutations of HCC have roles in predicting the clinical outcomes of the HCC transplant patients, exome sequencing was performed on the same HCC samples and their matched non-liver benign tissue samples from both cohorts. Somatic mutations were identified by subtracting the single-nucleotide variants in the cancer sample with the matched normal tissue from the same individual. A total of 30,090 somatic mutations were identified in 64 HCC samples of both cohorts, with an average 470 (15-2657) mutations per HCC sample (FIG. 20, Supplemental Table 2). These mutations were distributed in 6977 pathways based on Gene Ontology (GO) and Kyoto Encyclopedia of Genes and Genomes (KEGG). The difference of mutation numbers between the recurrence and non-recurrence cases in each pathway was ranked through t-tests. The pathway with the smallest p-value was ranked at the top. The top 5 mutation pathways were then combined with the 500 genes from the transcriptome sequencing to examine whether mutation status in the pathways improves the transcriptome prediction. This model was then added with top 10, 15, 20, 25, or 30 pathways to examine whether additional differential mutation pathways improve the prediction rate. The model with the best Youden Index was selected through LOOCV. As shown in FIGS. 4C and D, the combination of 5 mutation pathways and 500 differential expressed genes in the training set improved the accuracy to 86.8% with a sensitivity of 86.7% and specificity of 87% (AUC=0.87 and p=5.2×10₋₂₄). When this algorithm was applied to the testing set, the accuracy was 77% (AUC=0.764 and p=0.0065), with a sensitivity of 100% and specificity of 66.7% (FIG. 19, Supplemental Table 1). When both training and testing set data were combined to create a prediction model based on LOOCV method, the transcriptome model predicted 81.3% correctly (AUC=0.896 and p=2.2×10₋₂₄, FIG. 4E), while mutation pathways and transcriptome combination generated a correct prediction of 84.4% (AUC=0.894 and p=7.3×10⁻²⁴) with a sensitivity of 78.3% and specificity of 87.8% (FIG. 4F and FIG. 19, Supplemental Table 1).

Using transcriptome analysis alone, the survival analysis in the training set showed that 87% of the transplant patients predicted as non-recurrent enjoyed recurrence-free survival up to 298.8 months, while patients predicted as recurrence had a 20% 3-year recurrence-free survival rate (p=1.6×10⁻⁶, FIG. 5A). When the same algorithm from the training set was applied to the testing set, the patients predicted as non-recurrence had a 92.3% recurrence-free survival up to 60 months, while the patients predicted as recurrence had only about 46% recurrence-free survival in a similar period (p=0.01, FIG. 5B). The combination of transcriptome and mutation pathways analyses showed that the recurrence-free survival rates reached 90.9% for the patients predicted as non-recurrence in the training set and 100% in the testing set, while the patients predicted as recurrence had the recurrence-free survival rates of 18.8% (p=2.5×10⁻⁷) in the training set and 42.9% (p=0.002) in the testing set (FIG. 5C and 5D). These results suggest a mild improvement of the prediction of recurrence-free survival when mutation pathways analysis was added to the prediction model. When both training and testing cohorts were combined, similar mild improvement of survival prediction by transcriptome and mutation pathways model was shown: 87.8% patients predicted as non-recurrence by the transcriptome/mutation pathways RF model experienced at least 3 years of recurrence-free survival versus 85.4% by the transcriptome RF model, while only 21.7% patients predicted as recurrence by the transcriptome/mutation pathways RF model survived recurrence-free for the similar period versus 26.1% for the transcriptome RF model (FIG. 5E and 5F).

### Example 5

### Role of Milan Criteria in Predicting the Recurrence of HCC in the Transplant Patients

Milan criteria is a radiology-based parameter defined by the size and number of HCC tumor nodules. Based on Milan-in (low risk of recurrence) and Milan-out (high risk of recurrence) assessment, the prediction rate of the recurrence for the entire cohort is 76.6%, with a sensitivity of 78.2% and specificity of 75.6%. To investigate whether the addition of Milan criteria improves the prediction rate of the genome prediction model, the transcriptome/mutation pathways model and Milan score were combined to create a transcriptome/mutation pathways/Milan RF model to predict the likelihood of HCC recurrence of the liver transplant patients. As shown in FIGS. 9 and 10, even though the transcriptome/mutation pathways/Milan

RF model offered significant improvement of the prediction rates over the Milan criteria, the addition of the Milan criteria did not improve the prediction rate of transcriptome/mutation pathways RF model in the training analysis or training to testing analysis (FIG. 19, Supplemental Table 1).

To examine whether the other machine learning models were improved by Milan criteria, the transcriptome sequence results were analyzed through k-TSP method, a non-parametric algorithm especially suitable for cross-platform studies. The model provides a prediction score based on the k-top scoring pairs, where a positive value indicates recurrence and a negative score means non-recurrence prediction. The k-TSP model was applied to the training set for LOOCV with different numbers of top gene pairs (5, 7, 9 . . . , 49) and the best model was selected by the highest Youden Index. The transcriptome k-TSP model alone yielded 79% accuracy in the training analysis, 73.1% in the testing analysis and 79.7% in the combined training and testing analyses (FIGS. 11 and 12, and FIG. 19, Supplemental Table 1). The combination of Milan criteria and transcriptome sequencing produced significant improvement over either analysis alone (FIGS. 6 and 7): The Milan/transcriptome k-TSP model generated 79% prediction rate in the training analysis, 80.8% in the testing analysis, and 84.4% in the combined training and testing cross validation analysis. Interestingly, when DNA mutation pathway analysis was combined with the Milan/transcriptome k-TSP model, mixed results were obtained: The Milan/transcriptome/mutation pathways k-TSP model improved the prediction to 89.5% in the training set and 87.5% in the combined training and testing set, but dropped the prediction rate to 73.1% in the testing analysis (FIG. 19, Supplemental Table 1, FIGS. 11, 12 and 13). These results suggest that Milan criteria may improve the prediction of k-TSP machine learning model, particularly the k-TSP transcriptome analysis, when they were combined into an integrative prediction model.

Survival analysis showed that 94% HCC patients with Milan “in” enjoyed a recurrence-free survival of 3 years or more in the training set. However, the 3-year recurrence-free survival for Milan “in” patients decreased to 80% in the testing set and 86% in the combined data sets (FIG. 7(A-C)). The Milan/transcriptome k-TSP model showed a 90% 3-year survival rate in the training set when the patients were predicted as non-recurrence (FIG. 7D). The testing validation analysis showed that 83% HCC patients predicted by Milan/transcriptome k-TSP model as nonrecurrence survived up to 60 months without recurrence, while 37.5% patients predicted as recurrence survived similar periods without recurrence (p=0.016, FIG. 7E). When both training and testing cohorts were combined, the cancer-free survival improved to 85.7% for patients predicted as non-recurrence, and 22.7% for patients as recurrence (p=6.18×10⁻⁹, FIG. 7F), extremely similar to the survival results produced by transcriptome/mutation pathways/Milan RF model in the same data set: 85.7% HCC patients with 3 years recurrence-free survival when predicted as non-recurrence, while 22.7% patients with 3 years or longer cancer-free when predicted as recurrence (p=6.54×10⁻¹⁰). These may compare favorably with Milan criteria alone: 86% 3-year survival for Milan “in”, while 35.7% for Milan “out” (p=1.5×10⁻⁵, FIG. 10 (A-F)).

Next, the entire cohort was divided into low risk of recurrence (Milan-in) and high risk of recurrence (Milan-out) based on Milan criteria. The transcriptome/mutation pathways RF model was applied to predict the outcomes. When Milan is “in”, the model predicted 88.9% correctly based on the transcriptome/mutation pathways RF model (FIG. 19, Supplemental Table 1). Interestingly, when Milan is “out”, the model had an accuracy of 82.1%, with 94.4% sensitivity and 60% specificity, including predicting 17 of 18 recurrent patients correctly (FIG. 19, Supplemental Table 1). These results suggest that the genome model may have a significant utility in predicting the clinical outcomes of patients outside the Milan criteria.

### Example 6

### The Impact of Heterogeneity of HCC

HCC may have significant heterogeneity in terms of genome profile and differentiation even in the same individual (41). A tumor nodule may have different gene expression and mutation profiles from its nearby nodules. To investigate whether the genome prediction model is sufficiently robust to overcome the heterogeneous nature of HCC, 3 individuals with multiple tumor nodules were examined, including an individual (patient #1) having 4 tumor nodules and two individuals (patients #2 and #3) having 2 tumor nodules each. As shown in Table 2, the transcriptome/mutation pathways RF prediction model consistently produced scores indicating HCC recurrence from each of the four tumor nodules of patient #1, matching the clinical outcome of the patient. The transcriptome/mutation pathways RF model correctly predicted the non-recurrence outcomes from 2 tumor nodules of patient #2, while the same model predicted 2 tumor nodules of patient #3 as recurrence outcomes, matching the real clinical results. Of the eight tumor nodules, the genome prediction yielded a 100% (8/8) correct prediction. Overall, the genome prediction model is reasonably robust in predicting the clinical outcomes of HCC samples despite the heterogeneity of the cancers.

### Example 7

### The Signaling Pathways Involved in the Genome Prediction Model

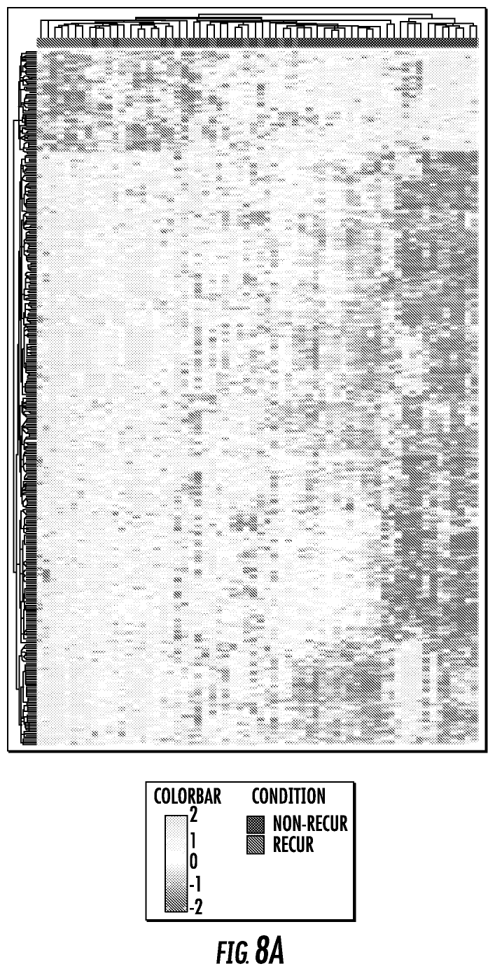

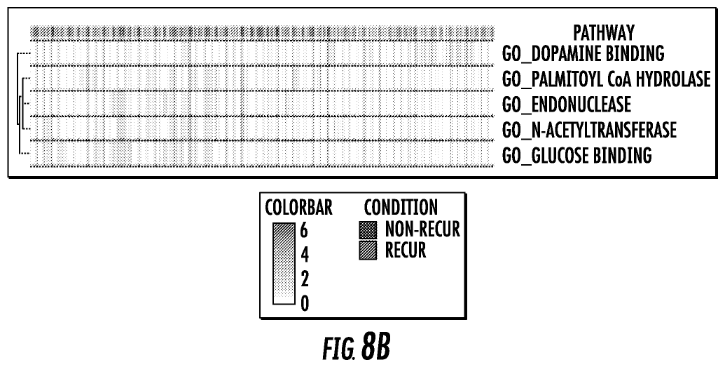

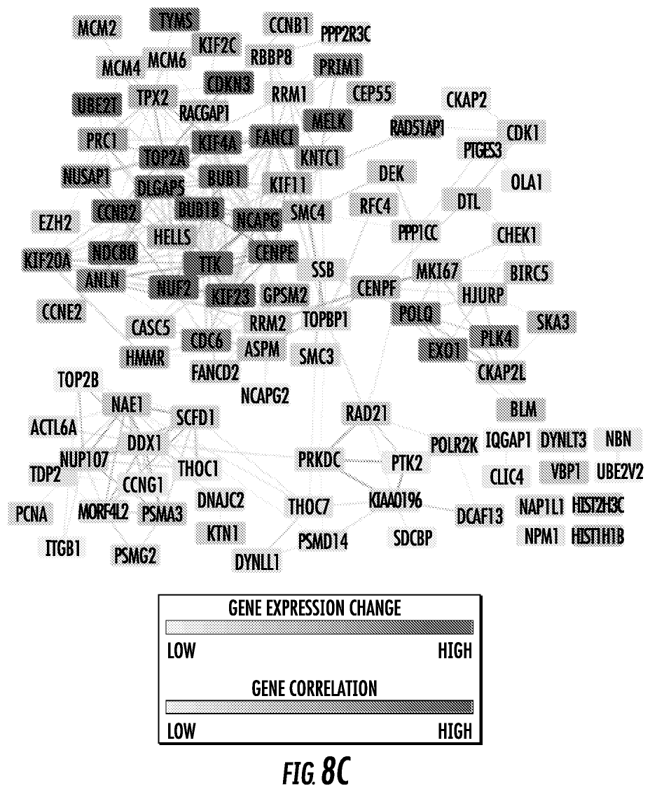

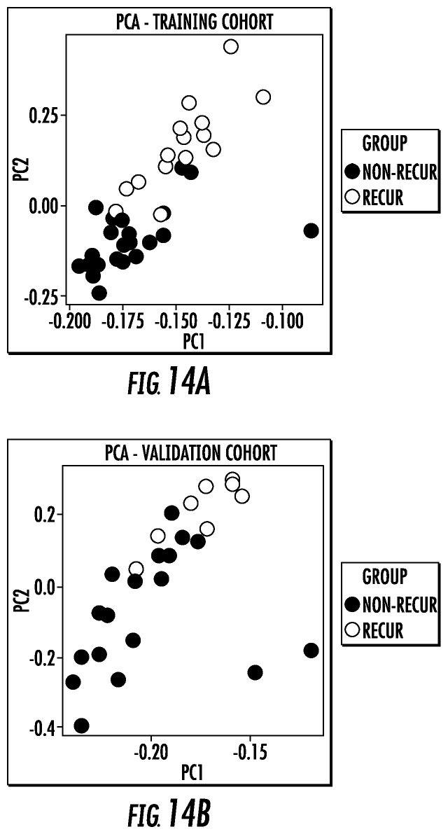

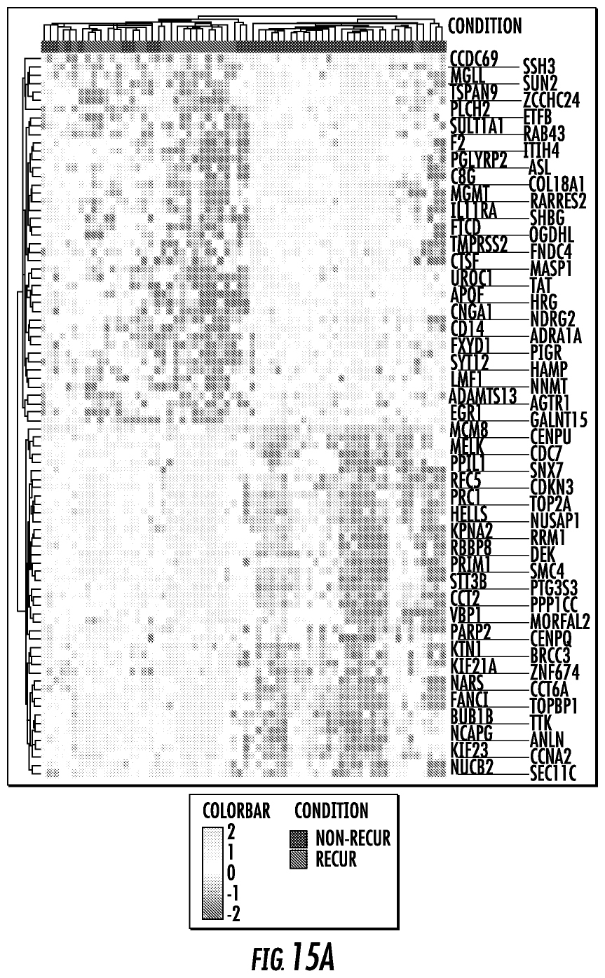

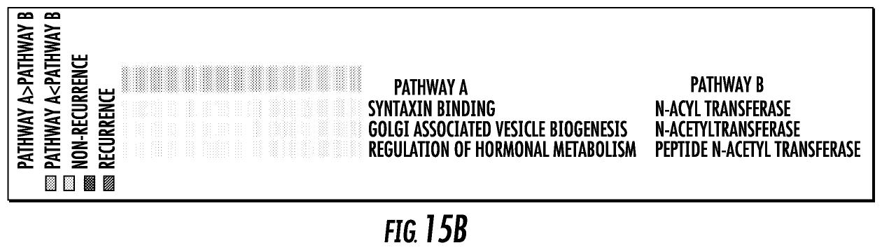

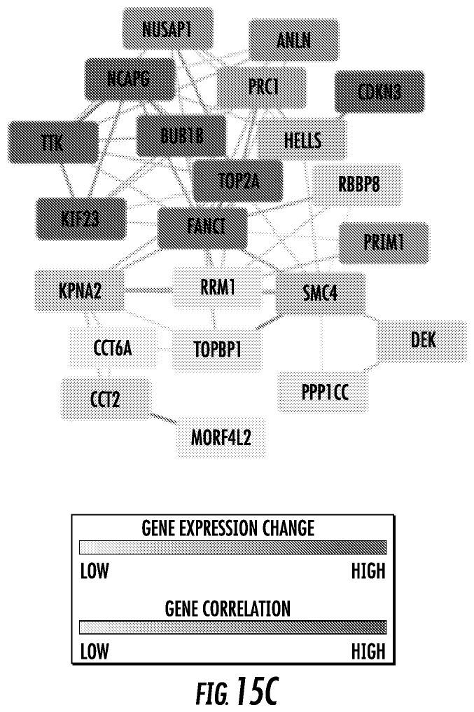

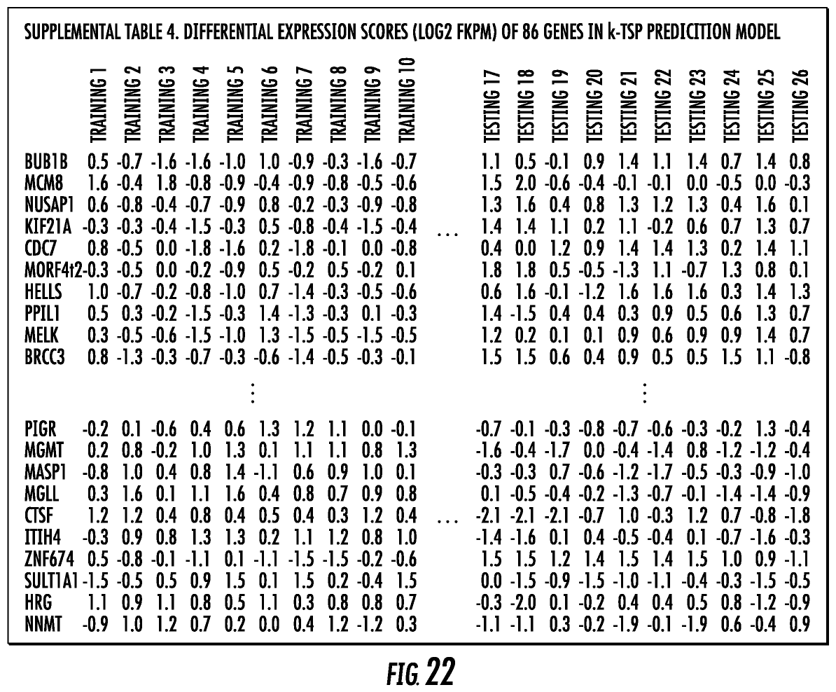

When the relative expression levels of the top 500 genes were used as parameters, most of the cancers with the non-recurrence outcomes appeared to aggregate together in the hierarchical clustering analysis (FIG. 8A) and principal component analysis (FIG. 14), separating from the samples with the recurrence outcomes. Similar segregation of recurrence and non-recurrence samples was also achieved when using the top 43 pairs of genes from the k-TSP model (FIG. 15A). At the DNA level, the mutations in the pathway of dopamine binding were dominant in the samples from patients with HCC recurrence in the RF analysis (FIG. 8B), while mutations in the pathways of Syntaxin binding, Golgi associated vesicle biogenesis, and the regulation of hormonal metabolic process were included in the k-TSP model (FIG. 15B). The disruption of these pathways may impact the homeostasis and metabolisms of the cancer cells. Thus, these alterations may impact the survival of the cancers. At the transcriptome level, 77 of 86 genes in the k-TSP model overlapped with those from the RF models (FIGS. 21 and 22, Supplemental Table 3 and 4). Genes involving in DNA replication, chromosome segregation and mitoses such as CDKN3, MCM8, MCM6, BUB1B, KIF23 and CDC6 dominated the pathway analyses (FIG. 8C, FIG. 15C and 16). The copy number gain or over-expression of these genes had been previously reported in human cancers (13, 42-44).

These changes may facilitate the DNA replication and growth of cancer cells.

### Example 8

### Discussion

Liver transplantation is one of the main approaches to treat liver cancers. It is particularly useful for HCC patients with late-stage cirrhosis or other non-functional liver conditions. Milan criteria have been a useful criterion for gauging the suitability of liver transplant in the last 25 years.

While most patients inside the Milan criteria experienced cancer-free recovery from the liver Transplant (45), the criteria were considered overly strict, and excluded many patients from receiving the liver transplant treatment (45). The current genome prediction model, whether in combination with Milan criteria or not, represents a new alternative to Milan criteria for the selection of liver transplant candidates. Two potential clinical scenarios can be adopted using the genome prediction model: First, Milan criteria is used as the first line of selection of patient candidates for the liver transplant. The patients with “Milan-in” status will be selected as primary candidates for the liver transplant, while the patients with “Milan-out” status will be screened through the genome prediction model for the suitability of the liver transplant. Second, Milan criteria can be integrated into the genome prediction model to screen all HCC candidates for the suitability of the liver transplant. In either scenario, the model may produce an improvement on Milan criteria alone.

Overfitting is one of the potential pitfalls of molecular prediction models. To overcome the potential overfitting issues, we had preselected the HCC cases into two unconnected cohorts based on the year of transplant surgery. The testing cohort represents an ongoing prospective analysis. To increase the robustness of the analysis, most samples in one cohort (training) were analyzed through Illumina HiSeq2500, while another (testing) were analyzed through NextSeq550. Due to the differences of the platforms, the read lengths of the sequencing were also different: HiSeq2500 platform was limited to 100 bases per read, while NextSeq550DX was 150 bases. The sequencings were performed in different time frames (2015-2017 for the training set, 2018-2020 for the testing set). Despite the non-connected nature of the cohorts, different sequencing platforms and different time frames, the variation in prediction accuracies between the two cohorts was consistently less than 10%, suggesting a good reproducibility of the model. The robustness of the genome prediction model is not limited to RF method. When other machine learning methods such as k-TSP were applied, Support Vector Machine, Linear Discriminant Analysis, or Logistics Regression, similar results were obtained (FIG. 19, Supplemental Table 1).

A surprising finding in the analysis presented here is that most of the frequent mutations of HCC such as TP53, CTNNB1 and TERT were not found to play important roles in predicting the behavior of HCC in liver transplant patients. Rather, mutations in dopamine signaling pathway such as dopamine receptors and G-protein coupled receptors were frequent in HCC patients who experienced recurrence after the liver transplant, while mutations in genes involving in glucose binding/metabolism such as HKDC1, G6PD, and endonuclease such as RNASE2, XRCC3, were more frequent in HCC patients who were less likely to have cancer recurrence. The altered functions of these proteins may have an impact on the survival and metabolism of the cancer cells. In contrast, the transcriptome analysis shows that the most altered expression genes are those involving DNA synthesis (MCM8, MCM6, TOP2A and CDC7), chromatin segregation (BUB1 and CDC6) and mitosis (NDC80 and PPP1CC) (FIG. 8C and FIG. 15C). The over-expression of these genes are directly related to cancer cell growth and proliferation. However, most of these genes were not mutated.

The relative irrelevance of the cancer driver mutations associated directly with the malignant behavior of HCC cells for predicting post-transplant recurrence is understandable. The HCC recurrence occurs after circulating HCC cells in the peripheral blood at the time of the transplantation procedure traverse through the circulation, survive the turbulent flow environment of the cardiac valves, proceed through the pulmonary circulation without attaching to the lungs, and finally lodge themselves in the new liver (46, 47). This may be a complicated process and the pathways operating within the cells have to be able to allow them to withstand the immune and shear/stress forces likely to be encountered. The pathways enabling these capabilities are not well understood, and the findings from the current study are likely to provide useful information as to their nature. The mutation and transcriptome analyses appear to uncover two different facets of the cancer genome: a qualitative alteration without much change in expression levels and a quantitative change without the alteration of quality. Each change may have an impact on the cancer cells and leads to recurrence and metastasis. Future dissection of these pathways may help to gain a better understanding of the cancer behavior.

### References

1. Yang JD, Hainaut P, Gores GJ, Amadou A, Plymoth A, Roberts LR. A global view of hepatocellular carcinoma: trends, risk, prevention and management. Nat Rev Gastroenterol Hepatol 2019; 16:589-604.
2. Siegel RL, Miller KD, Jemal A. Cancer statistics, 2020. CA Cancer J Clin 2020;70:7-30.
3. Brenner DA. Thomas E. Starzl: Transplantation pioneer. Proc Natl Acad Sci U S A 2017;114:10808-10809.
4. Roayaie S, Schwartz JD, Sung MW, Emre SH, Miller CM, Gondolesi GE, Krieger NR, et al. Recurrence of hepatocellular carcinoma after liver transplant: patterns and prognosis. Liver Transpl 2004;10:534-540.
5. de'Angelis N, Landi F, Carra MC, Azoulay D. Managements of recurrent hepatocellular carcinoma after liver transplantation: A systematic review. World J Gastroenterol 2015;21:11185-11198.
6. Sapisochin G, Goldaracena N, Laurence JM, Dib M, Barbas A, Ghanekar A, Cleary SP, et al. The extended Toronto criteria for liver transplantation in patients with hepatocellular carcinoma: A prospective validation study. Hepatology 2016;64:2077-2088.
7. Sapisochin G, Goldaracena N, Astete S, Laurence JM, Davidson D, Rafael E, Castells L, et al. Benefit of Treating Hepatocellular Carcinoma Recurrence after Liver Transplantation and Analysis of Prognostic Factors for Survival in a Large Euro-American Series. Ann Surg Oncol 2015;22:2286-2294.
8. Jain A, Reyes J, Kashyap R, Dodson SF, Demetris AJ, Ruppert K, Abu-Elmagd K, et al. Long-term survival after liver transplantation in 4,000 consecutive patients at a single center. Ann Surg 2000;232:490-500.
9. Poynard T, Naveau S, Doffoel M, Boudjema K, Vanlemmens C, Mantion G, Messner M, et al. Evaluation of efficacy of liver transplantation in alcoholic cirrhosis using matched and simulated controls: 5-year survival. Multi-centre group. J Hepatol 1999;30:1130-1137.
10. Yu YP, Lin F, Dhir R, Krill D, Becich MJ, Luo JH. Linear amplification of gene-specific cDNA ends to isolate full-length of a cDNA. Analytical Biochemistry 2001;292:297-301.
11. Luo JH, Yu YP, Cieply K, Lin F, Deflavia P, Dhir R, Finkelstein S, et al. Gene expression analysis of prostate cancers. Mol Carcinog 2002;33:25-35.
12. Yu YP, Landsittel D, Jing L, Nelson J, Ren B, Liu L, McDonald C, et al. Gene expression alterations in prostate cancer predicting tumor aggression and preceding development of malignancy. J Clin Oncol 2004;22:2790-2799.
13. Ren B, Yu G, Tseng GC, Cieply K, Gavel T, Nelson J, Michalopoulos G, et al. MCM7 amplification and overexpression are associated with prostate cancer progression. Oncogene 2006;25:1090-1098.
14. Yu G, Tseng GC, Yu YP, Gavel T, Nelson J, Wells A, Michalopoulos G, et al. CSR1 suppresses tumor growth and metastasis of prostate cancer. American Journal of Pathology 2006; 168:597-607.
15. Yu YP, Yu G, Tseng G, Cieply K, Nelson J, Defrances M, Zarnegar R, et al. Glutathione peroxidase 3, deleted or methylated in prostate cancer, suppresses prostate cancer growth and metastasis. Cancer Res 2007;67:8043-8050.
16. Yu YP, Ding Y, Chen Z, Liu S, Michalopoulos A, Chen R, Gulzar ZG, et al. Novel fusion transcripts associate with progressive prostate cancer. Am J Pathol 2014; 184:2840-2849.
17. Luo JH, Liu S, Tao J, Ren BG, Luo K, Chen ZH, Nalesnik M, et al. Pten-NOLCI fusion promotes cancers involving MET and EGFR signalings. Oncogene 2020.
18. Bolger AM, Lohse M, Usadel B. Trimmomatic: a flexible trimmer for Illumina sequence data. Bioinformatics 2014;30:2114-2120.
19. Kim D, Paggi JM, Park C, Bennett C, Salzberg SL. Graph-based genome alignment and genotyping with HISAT2 and HISAT-genotype. Nat Biotechnol 2019;37:907-915.
20. Trapnell C, Williams BA, Pertea G, Mortazavi A, Kwan G, van Baren MJ, Salzberg SL, et al. Transcript assembly and quantification by RNA-Seq reveals unannotated transcripts and isoform switching during cell differentiation. Nat Biotechnol 2010;28:511-515.
21. Li H, Durbin R. Fast and accurate short read alignment with Burrows-Wheeler transform. Bioinformatics 2009;25:1754-1760.
22. McKenna A, Hanna M, Banks E, Sivachenko A, Cibulskis K, Kernytsky A, Garimella K, et al. The Genome Analysis Toolkit: a MapReduce framework for analyzing next-generation DNA sequencing data. Genome Res;20:1297-1303.
23. Cortes C, Vapnik V. Support-vector networks. Machine Learning 1995;20:273-297.
24. Amit Y, Geman D. Shape quantization and recognition with randomized trees. Neural computation 1997;9.7.
25. Bauer E, Kohavi R. An empirical comparison of voting classification algorithms: Bagging, boosting, and variants. Machine Learning 1999;36:105-139.
26. McLachlan GJ. Discriminant analysis and statistical pattern recognition. Applied Probability & Statistics 2004:1-526.
27. Tolles J, Meurer WJ. Logistic Regression: Relating Patient Characteristics to Outcomes. JAMA 2016;316:533-534.
28. Shi P, Ray S, Zhu Q, Kon MA. Top scoring pairs for feature selection in machine learning and applications to cancer outcome prediction. BMC Bioinformatics 2011; 12:375.
29. Afsari B, Fertig EJ, Geman D, Marchionni L. switchBox: an R package for k-Top Scoring Pairs classifier development. Bioinformatics 2015;31:273-274.
30. Ashburner M, Ball CA, Blake JA, Botstein D, Butler H, Cherry JM, Davis AP, et al. Gene ontology: tool for the unification of biology. The Gene Ontology Consortium. Nat Genet 2000;25:25-29.
31. Kanehisa M, Goto S. KEGG: kyoto encyclopedia of genes and genomes. Nucleic Acids Res 2000;28:27-30.
32. Jassal B, Matthews L, Viteri G, Gong C, Lorente P, Fabregat A, Sidiropoulos K, et al. The reactome pathway knowledgebase. Nucleic Acids Res 2020;48: D498-D503.
33. Rouillard AD, Gundersen GW, Fernandez NF, Wang Z, Monteiro CD, McDermott MG, Ma'ayan A. The harmonizome: a collection of processed datasets gathered to serve and mine knowledge about genes and proteins. Database (Oxford) 2016;2016.
34. Gu Z, Eils R, Schlesner M. Complex heatmaps reveal patterns and correlations in multidimensional genomic data. Bioinformatics 2016;32:2847-2849.
35. Maag JLV. gganatogram: An R package for modular visualisation of anatograms and tissues based on ggplot2. F1000Res 2018;7:1576.
36. Shannon P, Markiel A, Ozier O, Baliga NS, Wang JT, Ramage D, Amin N, et al. Cytoscape: a software environment for integrated models of biomolecular interaction networks. Genome Res 2003; 13:2498-2504.
37. Robin X, Turck N, Hainard A, Tiberti N, Lisacek F, Sanchez JC, Muller M. pROC: an open-source package for R and S+ to analyze and compare ROC curves. BMC Bioinformatics; 12:77.
38. Yuan Tang, Horikoshi M, Li W. ggfortify: Unified Interface to Visualize Statistical Results of Popular R Packages. The R Journal 2016;8:474-485.
39. Luo JH, Ren B, Keryanov S, Tseng GC, Rao UN, Monga SP, Strom S, et al. Transcriptomic and genomic analysis of human hepatocellular carcinomas and hepatoblastomas. Hepatology 2006;44:1012-1024.
40. Nalesnik MA, Tseng G, Ding Y, Xiang GS, Zheng ZL, Yu Y, Marsh JW, et al. Gene deletions and amplifications in human hepatocellular carcinomas: correlation with hepatocyte growth regulation. Am J Pathol 2012; 180:1495-1508.
41. Fransvea E, Paradiso A, Antonaci S, Giannelli G. HCC heterogeneity: molecular pathogenesis and clinical implications. Cell Oncol 2009;31:227-233.
42. Yu S, Wang G, Shi Y, Xu H, Zheng Y, Chen Y. MCMs in Cancer: Prognostic Potential and Mechanisms. Anal Cell Pathol (Amst) 2020;2020:3750294.
43. He DM, Ren BG, Liu S, Tan LZ, Cieply K, Tseng G, Yu YP, et al. Oncogenic activity of amplified miniature chromosome maintenance 8 in human malignancies. Oncogene 2017;36:3629-3639.
44. Santarius T, Shipley J, Brewer D, Stratton MR, Cooper CS. A census of amplified and overexpressed human cancer genes. Nat Rev Cancer 2010; 10:59-64.
45. Pavel MC, Fuster J. Expansion of the hepatocellular carcinoma Milan criteria in liver transplantation: Future directions. World J Gastroenterol 2018;24:3626-3636.
46. Rejniak KA. Circulating Tumor Cells: When a Solid Tumor Meets a Fluid Microenvironment. Adv Exp Med Biol 2016;936:93-106.
47. Krog BL, Henry MD. Biomechanics of the Circulating Tumor Cell Microenvironment. Adv Exp Med Biol 2018; 1092:209-233.

1. A computer-implemented method comprising:
receiving gene expression data related to a liver tissue sample for a subject having a liver cancer;
inputting the gene expression data into a trained machine learning model; and
predicting, using the trained machine learning model, a risk of recurrence of the liver cancer in the subject after liver transplantation.

2. The computer-implemented method of claim 1, wherein the trained machine learning model is a supervised machine learning model.

3. The computer-implemented method of claim 1, wherein the trained machine learning model is configured to predict the risk of recurrence as a probability score.

4. The computer-implemented method of claim 1, wherein the trained machine learning model is configured to predict the risk of recurrence by classifying the subject into one of a plurality of categories.

5. The computer-implemented method of claim 1, wherein the trained machine learning model is a support vector machine (SVM), a random forest model, a logistic regression model, or a k-top scoring pairs (k-TSP) model.

6. The computer-implemented method of claim 1, wherein the gene expression data comprises respective gene expression levels for a top-n differentially expressed genes, where n is an integer greater or equal to 10.

7. The computer-implemented method of claim 6, wherein n is greater than or equal to 50.

8. The computer-implemented method of claim 6, wherein the top-n differentially expressed genes comprise one or more of HOOK1, EFCAB7, CDC7, NUF2, UBE2T, HELLS, RRM1, SYT12, KIF21A, RACGAP1, PRIM1, PTGES3, YEATS4, CCT2, PARPBP, PPP1CC, KNTC1, TMED2, CDKN3, DLGAP5, BUB1B, NUSAP1, CCNB2, KIF23, FANCI, PRC1, CDC6, TOP2A, KPNA2, NDC80, RBBP8, NARS, BUB1, TOPBP1, SMC4, NCAPG, CENPE, PLK4, CENPU, CENPQ, TTK, FBX05, ANLN, MELK, DYNLT3, ZNF674, KIF4A, AMMECR1, ZNF449, and BRCC3.

9. The computer-implemented method of claim 6, wherein the trained machine learning model is a random forest model.

10. The computer-implemented method of claim 1, wherein the gene expression data comprises respective gene expression levels for a top-q pairs of differentially expressed genes, where q is an integer greater or equal to 10.

11. The computer-implemented method of claim 10, wherein q is 43.

12. The computer-implemented method of claim 10, wherein the top-q pairs of differentially expressed genes comprise one or more of BUB1B and SSH3, MCM8 and OGDHL, NUSAP1 and FNDC4, KIF21A and RAB43, CDC7 and CNGA1, MORF4L2 and ETFB, HELLS and HAMP, PPIL1 and ZCCHC24, MELK and GALNT15, BRCC3 and CCDC69, CCT6A and ASL, CDKN3 and SYT12, RBBP8 and TMPRSS2, KIF23 and LMF1, KPNA2 and SUN2, SMC4 and FXYD1, PPP1CC and FTCD, NUCB2 and NDRG2, PARP2 and IL11RA, VBP1 and AGTR1, TOP2A and TSPAN9, KTN1 and COL18A1, NCAPG and ADAMTS13, STT3B and CD14, SEC11C and C8G, CCNA2 and ADRA1A, CENPQ and UROC1, TTK and PLCH2, FANCI and SHBG, DEK and EGR1, RFC5 and APOF, PTGES3 and TAT, SNX7 and PGLYRP2, CCT2 and PIGR, PRC1 and MGMT, NARS and MASP1, RRM1 and MGLL, TOPBP1 and CTSF, F2 and ITIH4, ANLN and ZNF674, PRIM1 and SULTIA1, RARRES2 and HRG, and CENPU and NNMT.

13. The computer-implemented method of claim 10, wherein the trained machine learning model is a k-TSP model.

14. The computer-implemented method of claim 1, further comprising:
receiving mutation data related to the liver tissue sample; and
inputting the mutation data into the trained machine learning model.

15. The computer-implemented method of claim 14, wherein the mutation data comprises a number of somatic mutations present in the liver tissue sample from the subject.

16. The computer-implemented method of claim 14, wherein the mutation data comprises a number of somatic mutations present in a top-m mutation pathways, where m is an integer greater or equal to 5.

17. The computer-implemented method of claim 16, wherein m is 5.

18. The computer-implemented method of claim 16, wherein the top-m mutation pathways comprise one or more of GO_ENDONUCLEASE_ACTIVITY_ACTIVE_WITH_EITHER_RIBO_OR_DEOXYRIBON UCLEIC_ACIDS_AND_PRODUCING_3_PHOSPHOMONOESTERS, GO_GLUCOSE_BINDING, GO_PALMITOYL_COA_HYDROLASE_ACTIVITY, GO_PEPTIDE_N_ACETYLTRANSFERASE_ACTIVITY, and GO_DOPAMINE_BINDING.

19. The computer-implemented method of claim 16, wherein the trained machine learning model is a random forest model.

20. The computer-implemented method of claim 14, wherein the mutation data comprises a number of somatic mutations present in a top-r mutation pathway pairs, where r is an integer greater or equal to 3.

21. The computer-implemented method of claim 20, wherein r is 3.

22. The computer-implemented method of claim 20, wherein the top-r mutation pathways pairs comprise one or more of GO_SYNTAXIN_BINDING and GO_N_ACYLTRANSFERASE_ACTIVITY, REACTOME_GOLGI_ASSOCIATED_VESICLE_BIOGENESIS and GO_N_ACETYLTRANSFERASE_ACTIVITY, and GO_REGULATION_OF_HORMONE_METABOLIC_PROCESS and GO_PEPTIDE_N_ACETYLTRANSFERASE_ACTIVITY.

23. The computer-implemented method of claim 20, wherein the trained machine learning model is a k-top scoring pairs (k-TSP) model.

24. The computer-implemented method of claim 14, wherein the trained machine learning model is a support vector machine (SVM), a random forest model, a logistic regression model, or a k-top scoring pairs (k-TSP) model.

25. The computer-implemented method of claim 1, further comprising:
receiving a radiology-based parameter related to the liver cancer; and
inputting the radiology-based parameter into the trained machine learning model.

26. The computer-implemented method of claim 25, wherein the radiology-based parameter is based on a size or number of tumor nodules associated with the liver cancer.

27. The computer-implemented method of claim 25, wherein the radiology-based parameter is Milan criteria.

28. The computer-implemented method of claim 25, wherein the trained machine learning model is a k-top scoring pairs (k-TSP) model.

29. The computer-implemented method of claim 25, wherein the trained machine learning model is a support vector machine (SVM), a random forest model, or a logistic regression model.

30. The computer-implemented method of claim 1, further comprising:
receiving mutation data related to the liver tissue sample and a radiology-based parameter related to the liver cancer; and
inputting the mutation data and the radiology-based parameter into the trained machine learning model.

31. The computer-implemented method of claim 30, wherein the trained machine learning model is a random forest model.

32. The computer-implemented method of claim 30, wherein the trained machine learning model is a support vector machine (SVM), a logistic regression model, or a k-top scoring pairs (k-TSP) model.

33. The computer-implemented method of claim 1, further comprising providing a treatment recommendation based on the prediction.

34. The computer-implemented method of claim 33, wherein the treatment recommendation is to perform a liver transplant procedure on the subject.

35. The computer-implemented method of claim 1, wherein the liver cancer is hepatocellular carcinoma (HCC).

36. A method comprising:
predicting a risk of recurrence of a liver cancer in a subject after liver transplantation according to claim 1;
recommending the subject as a candidate for a liver transplant procedure based on the prediction; and
performing the liver transplant procedure on the subject.

37. A system comprising:
a trained machine learning model; and
a computing device comprising a processor and a memory, the memory having computer-executable instructions stored thereon that, when executed by the processor, cause the processor to:
receive gene expression data related to a liver tissue sample for a subject having a liver cancer;
input the gene expression data into the trained machine learning model; and
receive a risk of recurrence of the liver cancer in the subject after liver transplantation, wherein the risk of recurrence is predicted by the trained machine learning model.

38. The system of claim 37, wherein the memory has further computer-executable instructions stored thereon that, when executed by the processor, cause the processor to:
receive mutation data related to the liver tissue sample; and
input the mutation data into the trained machine learning model.

39. The system of claim 37, wherein the memory has further computer-executable instructions stored thereon that, when executed by the processor, cause the processor to:
receive a radiology-based parameter related to the liver cancer; and
input the radiology-based parameter into the trained machine learning model.

40. The system of claim 37, wherein the trained machine learning model is configured to predict the risk of recurrence as a probability score, or predict the risk of recurrence by classifying the subject into one of a plurality of categories.

41. The system of claim 37, wherein the trained machine learning model is a support vector machine (SVM), a random forest model, a logistic regression model, or a k-top scoring pairs (k-TSP) model.

42. The system of claim 37, wherein the memory has further computer-executable instructions stored thereon that, when executed by the processor, cause the processor to provide a treatment recommendation based on the prediction.

43. The system of claim 42, wherein the treatment recommendation is to perform a liver transplant procedure on the subject.

44. The system of claim 37, wherein the liver cancer is hepatocellular carcinoma (HCC).
